SEQUENCE LISTING (34 sequences)

SEQ ID NO: 1 - DNA, 849 bp, Arabidopsis thaliana
atgagtgtgattggaagcaagagctgtattttctctgtggctagatacacccgagaaaatgagaaatcttcttgcttcacttctatcaacaagaaatcttcgcttgatctgagatttcccagaaatctggcgggagtttcctgcaaattctctggagaaaatccaggaacaaatggggtttctcttagctccaagaacaaaatggaggattacaatacagctatgaagagattgatgagaagcccttatgaatatcatcatgatctaggtatgaactatacattgataagagatgaacttatagttgggtcacagccacagaaacctgaggacatagatcacttgaagcaggaacagaatgttgcttacatacttaacttgcagcaggataaggacattgagtattggggaatcgatttggattccattgttagaagatgtaaggagcttggaatccgtcacatgagaaggcctgctaaagattttgatccactttcgttgagaagccaacttccaaaagctgtttcttctttggaatgggcggtttcagaaggtaaaggaagagtctatgtgcattgctcagccggattgggaagagctccaggggtttccattgcttatatgtattggttctgtgacatgaatcttaacacggcttatgacactttggtatcaaagcgtccatgtgggcctaacaaaggagccatccgtggtgcaacatatgatctggctaagaatgatccctggaaagagccctttgagagtctccctgagaacgcattcgaggacattgcagattgggaaaggaagttgattcaagaacgtgttcgggccctccgtggaacctga

SEQ ID NO: 2 - protein, 282 aa, Arabidopsis thaliana
MSVIGSKSCIFSVARYTRENEKSSCFTSINKKSSLDLRFPRNLAGVSCKFSGENPGTNGVSLSSKNKMEDYNTAMKRLMRSPYEYHHDLGMNYTLIRDELIVGSQPQKPEDIDHLKQEQNVAYILNLQQDKDIEYWGIDLDSIVRRCKELGIRHMRRPAKDFDPLSLRSQLPKAVSSLEWAVSEGKGRVYVHCSAGLGRAPGVSIAYMYWFCDMNLNTAYDTLVSKRPCGPNKGAIRGATYDLAKNDPWKEPFESLPENAFEDIADWERKLIQERVRALRGT

SEQ ID NO: 3 - DNA, 34 bp, Artificial Sequence
  primer LBb1Sail
gccttttcagaaatggataaatagccttgcttcc

SEQ ID NO: 4 - DNA, 22 bp, Artificial Sequence
  primer LBb1 Salk
gcgtggaccgcttgctgcaact

SEQ ID NO: 5 - DNA, 19 bp, Artificial Sequence
  primer DS3-2 (for GT10871)
ccggtatatcccgttttcg

SEQ ID NO: 6 - DNA, 21 bp, Artificial Sequence
  primer SAIL_595 F04 LP
atattgcggtgcaactttacg

SEQ ID NO: 7 - DNA, 21 bp, Artificial Sequence
  primer SAIL_595 F04 RP
ctgagcatttatcagttgggg

SEQ ID NO: 8 - DNA, 20 bp, Artificial Sequence
  primer At3g10940_fw1
tgtgattggaagcaagagct

SEQ ID NO: 9 - DNA, 22 bp, Artificial Sequence
  primer At3g10940_re1
ccgaacacgttcttgaatcaac

SEQ ID NO: 10 - DNA, 21 bp, Artificial Sequence
  primer Salk_102567 LP
aagctgatgcgtaatgaatcg

SEQ ID NO: 11 - DNA, 21 bp, Artificial Sequence
  primer Salk_102567 RP
gaaatccccaaacatcctcac

SEQ ID NO: 12 - DNA, 22 bp, Artificial Sequence
  primer PP2A_F01
ctcttacctgcggtaataactg

SEQ ID NO: 13 - DNA, 20 bp, Artificial Sequence
  primer PP2A_R01
catggccgtatcatgttctc

SEQ ID NO: 14 - DNA, 21 bp, Artificial Sequence
  primer At3g10940_F01
gacatgaatcttaacacggct

SEQ ID NO: 15 - DNA, 19 bp, Artificial Sequence
  primer At3g10940_R01
atcatatgttgcaccacgg

SEQ ID NO: 16 - DNA, 26 bp, Artificial Sequence
  primer LSF2pGFP2 fw (KpnI site added)
ggggtaccatgagtgtgattggaagc

SEQ ID NO: 17 - DNA, 24 bp, Artificial Sequence
  primer LSF2pGFP2 rev (KpnI site added)
ggggtaccggttccacggagggcc

SEQ ID NO: 18 - DNA, 30 bp, Artificial Sequence
  primer LSF2prom fw
gattgcattattgatttgttgctcttgtag

SEQ ID NO: 19 - DNA, 27 bp, Artificial Sequence
  primer LSF2prom rev
cgttctctatctctcgttcttcacctg

SEQ ID NO: 20 - DNA, 24 bp, Artificial Sequence
  primer LSF2 full length cDNA fw
atgagtgtgattggaagcaagagc

SEQ ID NO: 21 - DNA, 18 bp, Artificial Sequence
  primer LSF2 full length cDNA rev
tcaggttccacggagggc

SEQ ID NO: 22 - DNA, 35 bp, Artificial Sequence
  primer delta65-LSF2_fw
tttcatatgaacaaaatggaggattacaatacagc

SEQ ID NO: 23 - DNA, 31 bp, Artificial Sequence
  primer delta65-LSF2_rev
aaactcgagtcatcaggttccacggagggcc

SEQ ID NO: 24 - DNA, 37 bp, Artificial Sequence
  primer delta78-LSF2_fw
tttcatatgatgagaagcccttatgaatatcatcatg

SEQ ID NO: 25 - DNA, 31 bp, Artificial Sequence; identical to SEQ ID NO: 23
  primer delta78-LSF2_rev

SEQ ID NO: 26 - DNA, 26 bp, Artificial Sequence
  primer LSF2-CT fw
gaatgatccctgaaaagagccctttg

SEQ ID NO: 27 - DNA, 26 bp, Artificial Sequence
  primer LSF2-CT rev
caaagggctcttttcagggatcattc

SEQ ID NO: 28 - DNA, 40 bp, Artificial Sequence
  primer LSF2 C193S fw
ggtaaaggaagagtctatgtgcattcttcagccggattgg

SEQ ID NO: 29 - DNA, 40 bp, Artificial Sequence
  primer LSF2 C193S rev
ccaatccggctgaagaatgcacatagactcttcctttacc

SEQ ID NO: 30 - protein, 8 aa, Artificial Sequence
  peptide sequence from an LSF-2 protein identified in Arabidopsis thaliana
DFDPLSLR

SEQ ID NO: 31 - protein, 8 aa, Artificial Sequence
  peptide sequence from an LSF-2 protein identified in Arabidopsis thaliana
DFDPLSLR

SEQ ID NO: 32 - protein, 13 aa, Artificial Sequence
  peptide sequence from an LSF-2 protein identified in Arabidopsis thaliana
AVSSLEWAVSEGK

SEQ ID NO: 33 - protein, 19 aa, Artificial Sequence
  peptide sequence from an LSF-2 protein identified in Arabidopsis thaliana
DELIVGSQPQKPEDIDHLK

SEQ ID NO: 34 - protein, 6 aa, Artificial Sequence
  peptide sequence from an LSF-2 protein identified in Arabidopsis thaliana
KLIQER

(19) United States
(12) Patent Application Publication
(10) Pub. No.: US 2014/0283819 A1
(43) Pub. Date: Sep. 25, 2014

(54) PLANTS WITH DECREASED ACTIVITY OF A STARCH DEPHOSPHORYLATING ENZYME

(71) Applicant: BAYER CROPSCIENCE AG, Monheim am Rhein (DE)

(72) Inventors: Samuel C. Zeeman, Zurich (CH); Oliver Koetting, Zurich (CH); Diana Santelia, Uster (CH)

(21) Appl. No.: 14/350,287

(22) PCT Filed: Oct. 10, 2012
(86) PCT No.: PCT/EP2012/070018

§ 371 Date: Apr. 7, 2014

Related U.S. Application Data
(60) Provisional application No. 61/546,203, filed on Oct. 12, 2011.

(30) Foreign Application Priority Data
Oct. 12, 2011 (EP) 1118944.4

(51) Int. Cl. C12N 15/82; C12N 15/113; C08B 31/06

(52) U.S. Cl. CPC C12N 15/8245; C08B 31/066; C12N 15/1137
USPC 127/32; 435/419; 800/298; 800/284; 127/67; 536/23.2; 435/196; 536/24.5

(57) ABSTRACT

The present invention relates to plant cells and plants that are genetically modified, whereby the genetic modification leads to a decrease in the activity of a starch dephosphorylating LSF-2 protein in comparison to corresponding wild type plant cells or wild type plants that have not been genetically modified. The present invention also relates to means and methods for the manufacture of such plant cells and plants. These types of plant cells and plants synthesise a modified starch. Therefore, the present invention also concerns the starch synthesised from the plant cells and plants according to the invention, methods for the manufacture of this starch, and the manufacture of starch derivatives of this modified starch, as well as flours containing starches according to the invention.

In addition, the present invention relates to chimeric genes comprising nucleic acids encoding a starch dephosphorylating LSF-2 protein, vectors, host cells such as plant cells, and plants containing such chimeric genes.

The present invention relates to plant cells and plants that are genetically modified, whereby the genetic modification leads to a decrease in the activity of a starch dephosphorylating LSF-2 protein in comparison to corresponding wild type plant cells or wild type plants that have not been genetically modified. The present invention also relates to means and methods for the manufacture of such plant cells and plants. These types of plant cells and plants synthesise a modified starch. Therefore, the present invention also concerns the starch synthesised from the plant cells and plants according to the invention, methods for the manufacture of this starch, and the manufacture of starch derivatives of this modified starch, as well as flours containing starches according to the invention.

In addition, the present invention relates to chimeric genes comprising nucleic acids encoding a starch dephosphorylating LSF-2 protein, vectors, host cells such as plant cells, and plants containing such chimeric genes.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

With regard to the increasing importance currently attributed to vegetable constituents as renewable raw material sources, one of the tasks of biotechnological research is to endeavour to adapt these vegetable raw materials to suit the requirements of the processing industry. Furthermore, in order to enable regenerating raw materials to be used in as many areas of application as possible, it is necessary to achieve a large variety of materials.

Polysaccharide starch is made up of chemically uniform base components, the glucose molecules, but constitutes a complex mixture of different molecule forms, which exhibit differences with regard to the degree of polymerisation and branching, and therefore differ strongly from one another in their physical-chemical characteristics. Discrimination is made between amylose starch, an essentially unbranched polymer made from alpha-1,4-glycosidically linked glucose units, and the amylopectin starch, a branched polymer, in which the branches come about by the occurrence of additional alpha-1,6-glycosidic links. A further essential difference between amylose and amylopectin lies in the molecular weight. While amylose, depending on the origin of the starch, has a molecular weight of 5×10⁵-10⁶ Da, that of the amylopectin lies between 10⁷ and 10⁸ The two macromolecules can be differentiated by their molecular weight and their different physical-chemical characteristics, which can most easily be made visible by their different iodine bonding characteristics.

Amylose has long been looked upon as a linear polymer, consisting of alpha-1,4-glycosidically linked alpha-D-glucose monomers. In other studies, however, the presence of alpha-1,6-glycosidic branching points (ca. 0.1%) has been shown (Hizukuri and Takagi, Carbohydr. Res. 134, (1984), 1-10; Takeda et al., Carbohydr. Res. 132, (1984), 83-92).

The functional characteristics of starches, such as for example the solubility, the retrogradation behaviour, the water binding capacity, the film-forming characteristics, the viscosity, the gelatinisation characteristics, the freezing-thawing stability, the acid stability, the gel strength and the size of the starch grain, are affected amongst other things by the amylose/amylopectin ratio, the molecular weight, the pattern of the side chain distribution, the ion concentration, the lipid and protein content, the average grain size of the starch, the grain morphology of the starch etc. The functional characteristics of starch are also affected by the phosphate content, a non-carbon component of starch. Here, differentiation is made between phosphate, which is bonded covalently in the form of monoesters to the glucose molecules of the starch (described in the following as starch phosphate), and phosphate in the form of phospholipids associated with the starch.

Starch phosphorylation is the only known modification of starch to occur in vivo. The extent of phosphorylation varies from a relatively high level in potato tuber starch (0.5% of glucosyl units) to almost undetectable amounts in the cereal starches (Blennow et al. (2000), Int J of Biological Macromolecules 27:211-18). Besides other influences, high-phosphate starches have a very high swelling power, forming transparent, viscous and freeze-thaw stable pastes, which are desired in many applications (Santelia and Zeeman (2011), Curr Opin Biotechnol 22:271-80).

Certain maize mutations, for example, synthesise a starch with increased starch phosphate content (waxy maize 0.002% and high-amylose maize 0.013%), while conventional types of maize only have traces of starch phosphate. Similarly small amounts of starch phosphate are found in wheat (0.001%), while no evidence of starch phosphate has been found in oats and sorghum. Small amounts of starch phosphate have also been fount in rice mutations (waxy rice 0.003%), and in conventional types of rice (0.013%). Significant amounts of starch phosphate have been shown in plants, which synthesise tubers or root storage starch, such as tapioca (0.008%), sweet potato (0.011%), arrowroot (0.021%) or potato (0.089%) for example. The percentage values for the starch phosphate content quoted above refer to the dry weight of starch in each case, and have been determined by Jane et al. (1996, Cereal Foods World 41 (11), 827-832).

Starch phosphate can be present in the form of monoesters at the C-2, C-3 or C-6 position of polymerised glucose monomers (Takeda and Hizukuri, 1971, Starch/Starke 23, 267-272). The distribution of phosphate in starch synthesised by plants is generally characterised in that approximately 30% to 40% of residual phosphate at the C-3 position, and approximately 60% to 70% of the residual phosphate at the C-6 position, of the glucose molecule are covalently bonded (Blennow et al., 2000, Int. J. of Biological Macromolecules 27, 211-218). Blennow et al. (2000, Carbohydrate Polymers 41, 163-174) have determined a starch phosphate content, which is bonded in the C-6 position of the glucose molecules, for different starches such as, for example, potato starch (between 7.8 and 33.5 nMol per mg of starch, depending on the type), starch from different Curcuma species (between 1.8 and 63 nMol per mg), tapioca starch (2.5 nMol per mg of starch), rice starch (1.0 nMol per mg of starch), mung bean starch (3.5 nMol per mg of starch) and sorghum starch (0.9 nMol per mg of starch). These authors have been unable to show any starch phosphate bonded at the C-6 position in barley starch and starches from different waxy mutations of maize. Up to now, it has not been possible to establish a connection between the genotype of a plant and the starch phosphate content (Jane et al., 1996, Cereal Foods World 41 (11), 827-832). It is therefore currently not possible to affect the starch phosphate content in plants by means of breeding measures.

Previously, a protein has been described, which facilitates the introduction of covalent bonds of phosphate residues to the glucose molecules of starch. This protein has the enzymatic activity of an alpha-glucan-water dikinase (GWD1 or SEX1, E.C.: 2.7.9.4) (Ritte et al., 2002, PNAS 99, 7166-7171), is frequently described in the literature as R1, and is bonded to the starch grains of the storage starch in potato tubers (Lorberth et al., 1998, Nature Biotechnology 16, 473-477). In the reaction catalysed by R1, the educts alpha-1,4-glucan (starch), adenosintriphosphate (ATP) and water are converted to the products glucan-phosphate (starch phosphate), monophosphate and adenosine monophosphate. In doing so, the residual gamma phosphate of the ATP is transferred to water, and the residual beta phosphate of the ATP is transferred to the glucan (starch). R1 transfers the residual beta phosphate of ATP to the C-6 position of the glucose molecules of alpha-1,4-glucans in vitro (Ritte et al., 2006, FEBS Letters 580, 4872-4876). Another protein, phosphoglucan, water dikinase (PWD or OK1) phosphorolyates the C3 position of the glucose molecules of alpha-1,4-glucans. PWD acts on glucan chains pre-phosphorylated by GWD (Baunsgaard et al. (2005). Plant Journal 41:595-605; Kotting et al. (2005). Plant Physiol 137:242-52; Ritte et al., 2006, FEBS Letters 580, 4872-4876). Starch from Arabidopsis sex1 (gwd) null mutants is essentially phosphate-free, whereas starch from pwd mutants is only phosphorylated at C6-positions (Ritte et al., 2006, FEBS Letters 580, 4872-4876).

Mutants plants not producing one of the two proteins display impaired starch degradation, leading to a starch-excess (sex) phenotype, which is severe in sex1 and more moderate in pwd (Kotting et al. (2005). Plant Physiol 137:242-52; Yu et al. (2001). Plant Cell 13:1907-1918).

Removal of the phosphate groups, at both the C3- and C6-positions, by the phosphoglucan phosphatase SEX4 (for Starch EXcess 4) is also required for proper starch metabolism (Kotting et al. (2009). Plant Cell 21:334-46; Hejazi et al. (2010). Plant Physiol 152:711-22). Although phosphate groups promote the solubilization of the starch granule surface, they can also obstruct glucan hydrolytic enzymes as demonstrated for β-amylase, which removes maltosyl units sequentially from the non-reducing end of an α-1,4-linked glucan chain. This exoamylase is required for starch degradation but cannot degrade past a phosphate group (Fulton et al. (2008) The Plant Cell 20:1040-58; Takeda and Hizukuri (1981), Carbohydr. Res. 89, 174-178). Carbohydrate Research 89:174-78). This suggests interdependence between reversible starch phosphorylation and glucan hydrolysis (Edner et al. (2007). Plant Physiol 145:17-28; Kotting et al. (2009) Plant Cell 21:334-46); Hejazi et al. (2010). Plant Physiol 152:711-22). The SEX4 protein possesses a carbohydrate binding module (CBM) and a phosphatase domain of the dual-specificity (DSP) class. Both domains are required for activity towards soluble and insoluble phospho-glucan substrates (Hejazi et al. (2010). Plant Physiol 152:711-22; Gentry et al. (2007). The Journal of Cell Biology 178:477-88; Niityla et al. (2006). JBC 281:11825-18). sex4 mutants have impaired starch degradation causing the sex phenotype to develop over repeated diurnal cycles (Kotting et al. (2009). Plant Cell 21:334-46; Niityla et al. (2006). JBC 281:11825-18). The decreased glucan phosphatase activity (sex4) results in the accumulation of phospho-glucans, mostly in the form of soluble phospho-oligosaccharides released from starch granule surface by α-amylase 3 (AMY3) and the isoamylase 3. These phospho-oligosaccharides are below the limit of detection in the wild type (Kotting et al. (2009). Plant Cell 21:334-46).

Apart from the increase of the starch phosphate content in plants, there are no available ways of specifically influencing the phosphorylation of starch in plants, of modifying the phosphate distribution within the starch synthesised by plants and/or of further increasing the starch phosphate content.

The object of the present invention is therefore based on providing modified starches with altered phosphate content and/or modified phosphate distribution, as well as plant cells and/or plants, which synthesise such a modified starch, as well as means and methods for producing said plants and/or plant cells.

This problem is solved by the embodiments described in the claims.

The present invention therefore relates to genetically modified plant cells or plants, characterised in that they have a reduced activity of at least one LSF-2 protein in comparison with corresponding wild type plant cells that have not been genetically modified.

In conjunction with the present invention, the term “wild type plant cell” means that the plant cells concerned were used as starting material for the manufacture of the plant cells according to the invention, i.e. their genetic information, apart from the introduced genetic modification, corresponds to that of a plant cell according to the invention.

In conjunction with the present invention, the term “wild type plant” means that the plants concerned were used as starting material for the manufacture of the plants according to the invention, i.e. their genetic information, apart from the introduced genetic modification, corresponds to that of a plant according to the invention.

In conjunction with the present invention, the term “corresponding” means that, in the comparison of several objects, the objects concerned that are compared with one another have been kept under the same conditions. In conjunction with the present invention, the term “corresponding” in conjunction with wild type plant cell or wild type plant means that the plant cells or plants, which are compared with one another, have been raised under the same cultivation conditions and that they have the same (cultivation) age.

The term “reduced activity of at least one LSF-2 protein” within the framework of the present invention means a reduction in the expression of endogenous genes, which encode the LSF-2 protein(s), and/or a reduction in the quantity of LSF-2 protein(s) in the cells, and/or a reduction in the enzymatic activity of LSF-2 protein(s) in the cells, all compared to that of non-genetically modified (wildtype) plant cells or (wildtype) plants of the same species.

The reduction in the expression can be determined by measuring the quantity of transcripts coding for LSF-2 protein(s), for example; e.g. by way of Northern Blot analysis or RT-PCR. A reduction preferably means a reduction in the quantity of transcripts of at least 50%, preferably at least 70%, more preferably at least 85%, and most preferably at least 90% in comparison to corresponding plant cells or plants that have not been genetically modified. A reduction in the quantity of transcripts encoding an LSF-2 protein in some embodiments also means that plants or plant cells not genetically modified according to the invention, which exhibit detectable quantities of transcripts encoding an LSF-2 protein, do not show detectable quantities of transcripts encoding an LSF-2 protein following genetic modification according to the invention.

The reduction in the amount of LSF-2 protein, which results in a reduced activity of this protein in the plant cells or plants concerned, can, for example, be determined by immunological methods such as Western blot analysis, ELISA (Enzyme Linked Immuno Sorbent Assay) or RIA (Radio Immune Assay). Here, a reduction preferably means a reduction in the amount of LSF-2 protein in comparison with corresponding plant cells or plants that have not been genetically modified by at least 50%, in particular by at least 70%, preferably by at least 85% and particularly preferably by at least 90%. A reduction in the amount of LSF-2 protein also means that plants or plant cells not genetically modified according to the invention that have detectable LSF-2 protein activity do not exhibit a detectable LSF-2 protein activity following genetic modification according to the invention.

Methods for manufacturing antibodies, which react specifically with a certain protein, i.e. which bond specifically to said protein, and which can be used e.g. for detecting LSF-2 protein or for reducing its activity are known to the person skilled in the art (see, for example, Lottspeich and Zorbas (Eds.), 1998, Bioanalytik, Spektrum akad, Verlag, Heidelberg, Berlin, ISBN 3-8274-0041-4). The manufacture of such antibodies is offered by some companies (e.g. Eurogentec, Belgium) as a contract service.

Within the framework of the present invention, the term “LSF-2 protein” is to be understood to be a phosphoric acid monoester hydrolase (E.C. 3.1.3). Specifically LSF-2 protein is to be understood to mean a protein which dephosphorylates glucan substrates including, but not limited to starch, solubilized amylopectin, (purified) phospho-oligosaccharides or amylopectin. LSF-2 proteins preferably release phosphate groups bound at the C3-position of the glucose molecules of (native) starch. LSF-2 proteins do not release phosphate bound to the C6 position of (native) starch. LSF-2 proteins can be described as glucan C3-phosphate phosphatase or as starch C3-phosphate phosphatase.

A LSF-2 protein catalyses a reaction of the general scheme:

Alpha-1,4-glucan-3-phopshate+H₂0→>Alpha-1,4-glucan+inorganic

Known glucan- or starch dephosphorylating proteins (e.g. LSF-1 and SEX-4) comprise a phosphatase domain with dual specificity (DSP), a carbohydrate binding domain (CBM) and a previously unknown C-terminal domain (CT). DSP and CBM are both required for activity of the respective proteins (Hejazi et al. (2010) Plant Physiol 152:711-22); Gentry et al. (2007), J. Cell Biol. 178, 477-488. The Journal of cell biology 178:477-88; Niityla et al. (2006). JBC 281:11825-18). The CBM is located between DSP and CT. Known glucan- or starch dephosphorylating proteins further comprise a PDZ-like protein-protein interaction domain. LSF-2 proteins do not comprise a CBM. The PDZ-like protein-protein interaction domain is also not present in the amino acid sequence of LSF-2 proteins. Despite the lack of the CBM present in other glucan- or starch dephosphorylating proteins, LSF-2 binds to starch. The binding to starch of LSF-2 proteins is less tight compared to known glucan- or starch dephosphorylating proteins.

LSF-2 proteins are characterized in that they comprise a DSP domain. Amino acid residues 85-247 display the DSP of the LSF-2 protein shown under SEQ ID NO 2. The canonical DSP domain of LSF-2 possesses the conserved amino acid residue motif HCxxGxxRA/T (where x is any amino acid residue). The motif is represented by amino acids 192 to 200 in the sequence shown under SEQ ID NO 2. The conserved cysteine (amino acid residue C193 in SEQ ID NO 2) in this active site motif is essential for activity of LSF-2 proteins.

LSF-2 proteins further display a C-terminal domain (CT). Amino acid residues 248-282 display the CT of the LSF-2 protein shown under SEQ ID NO 2. (see FIG. 1A, 2A,B). Deletion of the CT domain leads to a protein being (entirely) insoluble. (FIG. 2C)

The amino acid sequence of LSF-2 proteins comprises a plastid target signal sequence. Amino acid residues 1-61 define the plastid target sequence for the sequence shown under SEQ ID NO 2.

A nucleic acid sequence encoding a LSF-2 protein is shown under SEQ ID NO. 1 and an amino acid sequence of a LSF-2 protein is shown under SEQ ID NO. 2. Further amino acid sequences derivable therefrom can be obtained from Arabidopsis thaliana (NCBI Ref. Seq.: NP_{—}566383.1), Arabidopsis lyrata (NCBI Ref. Seq.: XP_{—}002884823.1), Populus trichocarpa (NCBI Ref. Seq.: XP_{—}002325379.1), Ricinus communis (NCBI Ref. Seq.: XP_{—}002520846.1), Zea mays (GenBank Acc.: ACN26193.1), Sorghum bicolor (NCBI Ref. Seq.: XP_{—}002441816.1), Oryza sativa (GenBank Acc.: EEE52638.1), Oryza sativa (NCBI Ref. Seq.: NP_{—}001065571.1), Vitis vinifera (NCBI Ref. Seq.: XP_{—}002274406.1), Selaginella moellendorffii (NCBI Ref. Seq.: XP_{—}002989045.1), Volvox carteri (NCBI Ref. Seq.: XP_{—}002947089.1), Chlamydomonas reinhardtii (NCBI Ref. Seq.: XP_{—}001695121.1), Chlorella variabilis (GenBank Acc.: EFN51916.1), Ostreococcus tauri (NCBI Ref. Seq.: XP_{—}003075237.1), Ostreococcus lucimarinus (NCBI Ref. Seq.: XP_{—}001416085.1), Micromonas sp. (NCBI Ref. Seq.: XP_{—}002502442.1), Micromonas pusilla (NCBI Ref. Seq.: XP_{—}003056994.1).

Inhibition of specific dephosphorylation of starch at the C3-position e.g. by reducing or abolishing LSF-2 activity in plant cells or plants enables for the production of starch with an increased amount of starch phosphate. Furthermore, abolishing LSF-2 activity in plant cells or plants enables for the production of starch with a modified phosphate in the starch. This leads to the production of starch with a different ratio of C3:C6 phosphorylation, i.e. the ratio shifts in favour of the C3-position as compared to plants or plant cells wherein LSF-2 expression is not reduced or abolished. In other words, less C3 positions are dephosphorylated as compared to wild type plant cells or plants, meaning that the starch synthesized by plant cells or plants according to the invention comprise a higher level of C3 starch phosphate compared to starch synthesized to wild type plant cells or plants.

In conjunction with the present invention, the term “starch phosphate” is to be understood to mean phosphate groups covalently bonded to the glucose molecules of starch.

Different methods of determining the amount of starch phosphate are described. Preferably, the method of determining the amount of starch phosphate described by Ritte et al. (2000, Starch/Starke 52, 179-185) can be used. Particularly preferably, the determination of the amount of starch phosphate by means of ³¹P-NMR is carried out according to the method described by Kasemusuwan and Jane (1996, Cereal Chemistry 73, 702-707).

In conjunction with the present invention, the term “phosphorylated starch” or “P-starch” is to be understood to mean a starch, which contains starch phosphate.

The activity of an LSF-2 protein can be demonstrated, for example, by the methods as described in the materials and general methods section below.

In addition, reducing LSF-2 activity in plant cells or plants does not decrease the biomass of plant cells or plants. A decrease in biomass has been observed when other enzyme activities involved in starch degradation like GWD and PWD are reduced. On the contrary, plant cells and plants according to the invention show an increases the biomass obtainable upon cultivation of said plants (see e.g. FIG. 10A). The biomass of plant cells or plants according to the invention is increased by at least 20%, preferably by at least 25%, more preferably at least 27%, even more preferably at least 30%, most preferably at least 32% and in particular preferably by at least 34% when compared to non-genetically modified wildtype plant cells or plants.

Preferably the biomass of plant cells or plants according to the invention is increased by at most 100%, more preferably by at most 80%, even more preferably by at most 60%, most preferably by at most 50% and in particular preferred by at most 45 when compared to non-genetically modified wildtype plant cells or plants.

The term “biomass” in connection with the present invention means the fresh weight in kilogram (kg) of the whole plant or the fresh weight in kilogram (kg) of harvestable plant parts. Preferably the biomass is calculated on the fresh weight of the material of harvestable plant parts per acreage, preferably only the green plant parts, optionally further comprising roots.

Whereas certain plant cells according to the invention may be able to regenerate into complete plants, in some embodiments, said plant cells cannot further develop or regenerate into a complete plant.

In one embodiment, the genetic modification consists or the introduction of at least one foreign nucleic acid molecule into the genome of the plant cell.

In this context, the term “genetic modification” means the introduction of homologous and/or heterologous foreign nucleic acid molecules into the genome of a plant cell or into the genome of a plant, wherein said introduction of these molecules leads to a reduction in the activity of an LSF-2 protein.

The plant cells according to the invention or plants according to the invention are modified with regard to their genetic information by the introduction of a foreign nucleic acid molecule. The presence or the expression of the foreign nucleic acid molecule leads to a phenotypic change. Here, “phenotypic” change means preferably a measurable change of one or more functions of the cells. For example, the genetically modified plant cells according to the invention and the genetically modified plants according to the invention exhibit a reduction in the activity of an LSF-2 protein or comprise a modified starch due to the presence of or in the expression of the introduced nucleic acid molecule.

In conjunction with the present invention, the term “foreign nucleic acid molecule” is understood to mean such a molecule that either does not occur naturally in the corresponding wild type plant cells, or that does not occur naturally in the concrete spatial arrangement in wild type plant cells, or that is localised at a place in the genome of the wild type plant cell at which it does not occur naturally. Preferably, the foreign nucleic acid molecule is a recombinant molecule, which consists of different elements, the combination or specific spatial arrangement of which does not occur naturally in vegetable cells.

In principle, the foreign nucleic acid molecule can be any nucleic acid molecule, which causes a reduction in the activity of an LSF-2 protein in the plant cell or plant.

In conjunction with the present invention, the term “genome” is to be understood to mean the totality of the genetic material present in a vegetable cell. It is known to the person skilled in the art that, in addition to the cell nucleus, other compartments (e.g. plastids, mitochondria) also contain genetic material.

A large number of techniques are available for the introduction of DNA into a vegetable host cell. These techniques include the transformation of vegetable cells with T-DNA using Agrobacterium tumefaciens or Agrobacterium rhizogenes as the transformation medium, the fusion of protoplasts, injection, the electroporation of DNA, the introduction of DNA by means of the biolistic approach as well as other possibilities. The use of agrobacteria-mediated transformation of plant cells has been intensively investigated and adequately described in EP 120516; Hoekema, IN: The Binary Plant Vector System Offsetdrukkerij Kanters B. V., Alblasserdam (1985), Chapter V; Fraley et al., Crit. Rev. Plant Sci. 4, 1-46 and by An et al. EMBO J. 4, (1985), 277-287. For the potato transformation, see Rocha-Sosa et al., EMBO J. 8, (1989), 29-33, for example.

The transformation of monocotyledonous plants by means of vectors based on Agrobacterium transformation has also been described (Chan et al., Plant Mol. Biol. 22, (1993), 491-506; Hiei et al., Plant J. 6, (1994) 271-282; Deng et al, Science in China 33, (1990), 28-34; Wilmink et al., Plant Cell Reports 11, (1992), 76-80; May et al., Bio/Technology 13, (1995), 486-492; Conner and Domisse, Int. J. Plant Sci. 153 (1992), 550-555; Ritchie et al, Transgenic Res. 2, (1993), 252-265). An alternative system to the transformation of monocotyledonous plants is transformation by means of the biolistic approach (Wan and Lemaux, Plant Physiol. 104, (1994), 37-48; Vasil et al., Bio/Technology 11 (1993), 1553-1558; Ritala et al., Plant Mol. Biol. 24, (1994), 317-325; Spencer et al., Theor. Appl. Genet. 79, (1990), 625-631), protoplast transformation, electroporation of partially permeabilised cells and the introduction of DNA by means of glass fibres. In particular, the transformation of maize has been described in the literature many times (cf. e.g. WO95/06128, EP0513849, EPO465875, EP0292435; Fromm et al., Biotechnology 8, (1990), 833-844; Gordon-Kamm et al., Plant Cell 2, (1990), 603-618; Koziel et al., Biotechnology 11 (1993), 194-200; Moroc et al., Theor. Appl. Genet. 80, (1990), 721-726).

The successful transformation of other types of cereal has also already been described, for example for barley (Wan and Lemaux, see above; Ritala et al., see above; Krens et al., Nature 296, (1982), 72-74) and for wheat (Nehra et al., Plant J. 5, (1994), 285-297; Becker et al., 1994, Plant Journal 5, 299-307). All the above methods are suitable within the framework of the present invention.

Amongst other things, plant cells and plants, which have been genetically modified by the introduction of an LSF-2 protein, can be differentiated from wild type plant cells and wild type plants respectively in that they contain a foreign nucleic acid molecule, which does not occur naturally in wild type plant cells or wild type plants, or in that such a molecule is present integrated at a place in the genome of the plant cell according to the invention or in the genome of the plant according to the invention at which it does not occur in wild type plant cells or wild type plants, i.e. in a different genomic environment. Furthermore, plant cells according to the invention and plants according to the invention of this type differ from wild type plant cells and wild type plants respectively in that they contain at least one copy of the foreign nucleic acid molecule stably integrated within their genome, possibly in addition to naturally occurring copies of such a molecule in the wild type plant cells or wild type plants. If the foreign nucleic acid molecule(s) introduced into the plant cells according to the invention or into the plants according to the invention is (are) additional copies of molecules already occurring naturally in the wild type plant cells or wild type plants respectively, then the plant cells according to the invention and the plants according to the invention can be differentiated from wild type plant cells or wild type plants respectively in particular in that this additional copy or these additional copies is (are) localised at places in the genome at which it does not occur (or they do not occur) in wild type plant cells or wild type plants. This can be verified, for example, with the help of a Southern blot analysis.

Furthermore, the plant cells according to the invention and the plants according to the invention can preferably be differentiated from wild type plant cells or wild type plants respectively by at least one of the following characteristics: If the foreign nucleic acid molecule that has been introduced is heterologous with respect to the plant cell or plant, then the plant cells according to the invention or plants according to the invention have transcripts of the introduced nucleic acid molecules. These can be verified, for example, by Northern blot analysis or by RT-PCR (Reverse Transcription Polymerase Chain Reaction). Plant cells according to the invention and plants according to the invention, which express an antisense and/or an RNAi transcript, can be verified, for example, with the help of specific nucleic acid probes, which are complimentary to the RNA (occurring naturally in the plant cell), which is coding for the protein. Preferably, the plant cells according to the invention and the plants according to the invention contain a protein, which is encoded by an introduced nucleic acid molecule. This can be demonstrated by immunological methods, for example, in particular by a Western blot analysis. In a further embodiment of the invention, the foreign nucleic acid molecule encodes a protein having the activity of a LSF-2 protein or the nucleic acid molecule is a part of a nucleic acid molecule encoding a LSF-2 protein or the foreign nucleic acid molecule is complementary to any of a sequence just mentioned. Example sequences of proteins which may have LSF-2 activity are listed elsewhere in this application.

In a further embodiment, the present invention relates to plant cells according to the invention and plants according to the invention, wherein said foreign nucleic acid molecule is selected from the group consisting of (a) DNA molecules, which encode at least one antisense RNA, which effects a reduction in the expression of at least one endogenous gene, which encodes an LSF-2 protein; (b) DNA molecules, which by means of a co-suppression effect lead to the reduction in the expression of at least one endogenous gene, which encodes an LSF-2 protein; (c) DNA molecules, which encode at least one ribozyme, which splits specific transcripts of at least one endogenous gene, which encodes an LSF-2 protein; (d) DNA molecules, which simultaneously express at least one antisense RNA and at least one sense RNA, wherein the said antisense RNA and the said sense RNA form a double-stranded RNA molecule, which effects a reduction in the expression of at least one endogenous gene, which encodes an LSF-2 protein (RNAi technology); (e) Nucleic acid molecules introduced by means of in vivo mutagenesis, which lead to a mutation or an insertion of a heterologous sequence in at least one endogenous gene encoding an LSF-2 protein, wherein the mutation or insertion effects a reduction in the expression of a gene encoding an LSF-2 protein or results in the synthesis of inactive LSF-2 proteins; (f) Nucleic acid molecules, which encode an antibody, wherein the antibody results in a reduction in the activity of an LSF-2 protein due to the bonding to an LSF-2 protein; (g) DNA molecules, which contain transposons, wherein the integration of these transposons leads to a mutation or an insertion in at least one endogenous gene encoding an LSF-2 protein, which effects a reduction in the expression of at least one gene encoding an LSF-2 protein, or results in the synthesis of inactive LSF-2 proteins; or (h) T-DNA molecules, which, due to insertion in at least one endogenous gene encoding an LSF-2 protein, effect a reduction in the expression of at least one gene encoding an LSF-2 protein, or result in the synthesis of inactive LSF-2 protein.

Inhibitory RNA molecules decrease the levels of mRNAs of their target expression products such as target proteins available for translation into said target protein. In this way, expression of proteins, for example those involved in stomatal opening or closing (aperture), can be inhibited. This can be achieved through well established techniques including co-suppression (sense RNA suppression), antisense RNA, double-stranded RNA (dsRNA), or microRNA (miRNA).

A DNA molecule encoding an RNA molecule as disclosed herein comprises a part of a nucleotide sequence encoding LSF-2 protein or a homologous sequence to down-regulate the expression of said LSF-2. Another example for an RNA molecule for use in down-regulating expression are antisense RNA molecules comprising a nucleotide sequence complementary to at least a part of a nucleotide sequence encoding LSF-2 or a homologous sequence. Here, down-regulation may be effected e.g. by introducing this antisense RNA or a chimeric DNA encoding such RNA molecule. In yet another example, expression of LSF-2 is down-regulated by introducing a DNA molecule encoding a double-stranded RNA molecule comprising a sense and an antisense RNA region corresponding to and respectively complementary to at least part of a gene sequence encoding said expression product of interest, which sense and antisense RNA region are capable of forming a double stranded RNA region with each other. Such double-stranded RNA molecule may be encoded both by sense and antisense molecules as described above and by a single-stranded molecule being processed to form siRNA (as described e.g. in EP1583832) or miRNA.

Furthermore, the use of introns, i.e. of non-coding areas of genes, which code for LSF-2 proteins, is also conceivable for achieving an antisense or a co-suppression effect. The use of intron sequences for inhibiting the gene expression of genes, which code for starch biosynthesis proteins, has been described in the international patent applications WO97/04112, WO97/04113, WO98/37213, WO98/37214.

In one example, expression of a target protein may be down-regulated by introducing a DNA molecule which encodes a sense RNA molecule capable of down-regulating expression of LSF-2 by co-suppression. The transcribed DNA region will yield upon transcription a so-called sense RNA molecule capable of reducing the expression of a gene encoding LSF-2 in the target plant or plant cell in a transcriptional or post-transcriptional manner. The transcribed DNA region (and resulting RNA molecule) comprises at least 20 consecutive nucleotides having at least 95% sequence identity to the corresponding portion of the nucleotide sequence encoding the target expression product such as a target protein present in the plant cell or plant.

Alternatively, a DNA molecule might encode an antisense RNA molecule. Down-regulating or reducing the expression of LSF-2 in the target plant or plant cell is effected in a transcriptional or post-transcriptional manner. The transcribed DNA region (and resulting RNA molecule) comprises at least 20 consecutive nucleotides having at least 95% sequence identity to the complement of the corresponding portion of the nucleic acid sequence encoding said target expression product present in the plant cell or plant.

However, the minimum nucleotide sequence of the antisense or sense RNA region of about 20 nt of the DNA molecule encoding the inhibitory RNA may be comprised within a larger RNA molecule, varying in size from 20 nt to a length equal to the size of the target gene. The mentioned antisense or sense nucleotide regions may thus be about from about 21 nt to about 5000 nt long, such as 21 nt, 40 nt, 50 nt, 100 nt, 200 nt, 300 nt, 500 nt or 1000 nt or larger in length. Moreover, it is not required for the purpose of the invention that the nucleotide sequence of the used inhibitory RNA molecule or the encoding region of the transgene, is completely identical or complementary to the target gene, i.e. the LSF-2 gene the expression of which is targeted to be reduced in the plant cell. The longer the sequence, the less stringent the requirement for the overall sequence identity is. Thus, the sense or antisense regions may have an overall sequence identity of about 40% or 50% or 60% or 70% or 80% or 90% or 95% or 98% or 100% to the nucleotide sequence of the target gene or the complement thereof. However, as mentioned, antisense or sense regions should comprise a nucleotide sequence of 20 consecutive nucleotides having about 95 to about 100% sequence identity to the nucleotide sequence encoding the target gene. The stretch of about 95 to about 100% sequence identity may be about 50, 75 or 100 nt.

The efficiency of the above mentioned chimeric genes for antisense RNA or sense RNA-mediated gene expression level down-regulation may be further enhanced by inclusion of DNA elements which result in the expression of aberrant, non-polyadenylated inhibitory RNA molecules. One such DNA element suitable for that purpose is a DNA region encoding a self-splicing ribozyme, as described in WO 00/01133. The efficiency may also be enhanced by providing the generated RNA molecules with nuclear localization or retention signals as described in WO 03/076619.

In addition, an expression product as described herein may be a DNA molecule which yields a double-stranded RNA molecule capable of down-regulating expression of an LSF-2 gene. Upon transcription of the DNA region the RNA is able to form dsRNA molecule through conventional base paring between a sense and antisense region, whereby the sense and antisense region are nucleotide sequences as hereinbefore described. Expression products being dsRNA according to the invention may further comprise an intron, such as a heterologous intron, located e.g. in the spacer sequence between the sense and antisense RNA regions in accordance with the disclosure of WO 99/53050. To achieve the construction of such a transgene, use can be made of the vectors described in WO 02/059294 A1.

In an example, said DNA molecule encodes an RNA molecule comprising a first and second RNA region wherein 1. said first RNA region comprises a nucleotide sequence of at least 19 consecutive nucleotides having at least about 94% sequence identity to the nucleotide sequence of said gene comprised in said cotton plant; 2. said second RNA region comprises a nucleotide sequence complementary to said 19 consecutive nucleotides of said first RNA region; 3. said first and second RNA region are capable of base-pairing to form a double stranded RNA molecule between at least said 19 consecutive nucleotides of said first and second region.

Another example inhibitory RNA to be encoded by a DNA molecule is a microRNA molecule (miRNA, which may be processed from a pre-microRNA molecule) capable of guiding the cleavage of mRNA transcribed from the DNA encoding LSF-2, which is to be translated into LFS-2 protein. miRNA molecules or pre-miRNA molecules may be conveniently introduced into plant cells through expression from a chimeric gene as described herein below comprising a (second) nucleic acid sequence encoding as expression product of interest such miRNA, pre-miRNA or primary miRNA transcript.

miRNAs are small endogenous RNAs that regulate gene expression in plants, but also in other eukaryotes. As used herein, a “miRNA” is an RNA molecule of about 19 to 22 nucleotides in length which can be loaded into a RISC complex and direct the cleavage of a target RNA molecule, wherein the target RNA molecule comprises a nucleotide sequence essentially complementary to the nucleotide sequence of the miRNA molecule. In one example, one or more of the following mismatches may occur in the essentially complementary sequence of the miRNA molecule:
A mismatch between the nucleotide at the 5′ end of said miRNA and the corresponding nucleotide sequence in the target RNA molecule;
A mismatch between any one of the nucleotides in position 1 to position 9 of said miRNA and the corresponding nucleotide sequence in the target RNA molecule;
Three mismatches between any one of the nucleotides in position 12 to position 21 of said miRNA and the corresponding nucleotide sequence in the target RNA molecule provided that there are no more than two consecutive mismatches;
No mismatch is allowed at positions 10 and 11 of the miRNA (all miRNA positions are indicated starting from the 5′ end of the miRNA molecule).

As used herein, a “pre-miRNA” molecule is an RNA molecule of about 100 to about 200 nucleotides, preferably about 100 to about 130 nucleotides which can adopt a secondary structure comprising a dsRNA stem and a single stranded RNA loop and further comprising the nucleotide sequence of the miRNA and its complement sequence of the miRNA* in the double-stranded RNA stem. Preferably, the miRNA and its complement are located about 10 to about 20 nucleotides from the free ends of the miRNA dsRNA stem. The length and sequence of the single stranded loop region are not critical and may vary considerably, e.g. between 30 and 50 nt in length. Preferably, the difference in free energy between unpaired and paired RNA structure is between −20 and −60 kcal/mole, particularly around −40 kcal/mole. The complementarity between the miRNA and the miRNA* does not need to be perfect and about 1 to 3 bulges of unpaired nucleotides can be tolerated. The secondary structure adopted by an RNA molecule can be predicted by computer algorithms conventional in the art such as mFold, UNAFoId and RNAFoId. The particular strand of the dsRNA stem from the pre-miRNA which is released by DCL activity and loaded onto the RISC complex is determined by the degree of complementarity at the 5′ end, whereby the strand which at its 5′ end is the least involved in hydrogen bonding between the nucleotides of the different strands of the cleaved dsRNA stem is loaded onto the RISC complex and will determine the sequence specificity of the target RNA molecule degradation. However, if empirically the miRNA molecule from a particular synthetic pre-miRNA molecule is not functional because the “wrong” strand is loaded on the RISC complex, it will be immediately evident that this problem can be solved by exchanging the position of the miRNA molecule and its complement on the respective strands of the dsRNA stem of the pre-miRNA molecule. As is known in the art, binding between A and U involving two hydrogen bounds, or G and U involving two hydrogen bounds is less strong that between G and C involving three hydrogen bounds.

miRNA molecules may be comprised within their naturally occurring pre-miRNA molecules but they can also be introduced into existing pre-miRNA molecule scaffolds by exchanging the nucleotide sequence of the miRNA molecule normally processed from such existing pre-miRNA molecule for the nucleotide sequence of another miRNA of interest. The scaffold of the pre-miRNA can also be completely synthetic. Likewise, synthetic miRNA molecules may be comprised within, and processed from, existing pre-miRNA molecule scaffolds or synthetic pre-miRNA scaffolds.

Example DNA molecules can also encode ribozymes catalyzing either their own cleavage or the cleavage of other RNAs.

Mutations in a nucleotide sequence, particularly in the protein encoding nucleotide sequence of a gene can be conveniently made by generating a double stranded break in such nucleotide sequence and allowing the ends to be rejoined by non-homologous end joining (NHEJ). Imprecise joining of the ends may lead to the loss of nucleotides resulting in frame shift mutations leading to nonsense translated products. Occasionally, small insertions of one to a few mutations may also occur. See e.g. Curtin et al. Plant Physiol. 2011 June; 156(2):466-73.

Therefore, the present invention further comprises a method for inducing a mutation in a gene encoding a protein with the activity of an LSF-2 protein in the genome of a plant cell or plant, comprising the steps of
a) providing a rare-cleaving double stranded break inducing enzyme to said plant cell wherein said rare cleaving double stranded break inducing enzyme specifically recognizes a sequence comprising 10 to 50 consecutive nucleotides selected from SEQ ID N-1;
b) selecting those plant cells or plants wherein the nucleotide sequence of said gene encoding a protein with the activity of an LSF-2 protein has been altered, and preferably wherein expression of said gene encoding a protein with the activity of an LSF-2 protein is inactivated or suppressed.

As used herein, a “double stranded DNA break inducing rare-cleaving endonuclease” is an enzyme capable of inducing a double stranded DNA break at a particular nucleotide sequence, called the “recognition site”. Rare-cleaving endonucleases, also sometimes called mega-nucleases, have a recognition site of 14 to 40 consecutive nucleotides. Therefore, rare-cleaving endonucleases have a very low frequency of cleaving, even in the larger plant genomes.

The double stranded DNA breaks in the transforming DNA molecule may be induced conveniently by transient introduction of a plant-expressible chimeric gene comprising a plant-expressible promoter region operably linked to a DNA region encoding a double stranded break inducing enzyme. The endonuclease itself, as a protein, could also be introduced into the plant cells, e.g. by electroporation. However, the endonuclease can also be provided in a transient manner by introducing into the genome of a plant cell or plant, a chimeric gene comprising the endonuclease coding region operably linked to an inducible plant-expressible promoter, and providing the appropriate inducible compound for a limited time. The endonuclease could also be provided as an RNA precursor encoding the endonuclease.

The double stranded break at the desired location in the nucleotide sequence of interest can be induced by provision of a rare-cleaving double stranded break inducing enzyme, which has been tailored to recognize a subsequence of the nucleotide of interest. Several techniques for generating such custom made double stranded break inducing enzymes that recognize basically any target nucleotide sequence of choice are available in the art.

Chimeric restriction enzymes can be prepared using hybrids between a zinc-finger domain designed to recognize a specific nucleotide sequence and the non-specific DNA-cleavage domain from a natural restriction enzyme, such as Fokl. Such methods have been described e.g. in WO 03/080809, WO94/18313 or WO95/09233 and in Isalan et al., 2001, Nature Biotechnology 19, 656-660; Liu et al. 1997, Proc. Natl. Acad. Sci. USA 94, 5525-5530).

Another way of producing custom double stranded break inducing enzymes is by re-iterative selection from a library of variants of homing endonucleases such as I-CreI, as described e.g. in WO2004/067736.

Yet another possibility to generate tailor made rare cleaving double stranded break inducing enzymes is by creating so-called TALE nucleases, by creating a DNA binding domain based on the modular transcription activator like effector proteins from pathogens, using the information and techniques described in WO2010/079430, and linking such DNA binding domain to the cleaving domain of a TypeII restriction endonuclease, such as Fok I, as described in WO2011/072246.

In conjunction with the present invention, plant cells and plants according to the invention can also be manufactured by the use of so-called insertion mutagenesis (overview article: Thorneycroft et al., 2001, Journal of experimental Botany 52 (361), 1593-1601). Insertion mutagenesis is to be understood to mean particularly the insertion of transposons or so-called transfer DNA (T-DNA) into a gene or near a gene coding for an LSF-2 protein, whereby, as a result of which, the activity of an LSF-2 protein in the cell concerned is reduced.

The transposons can be both those that occur naturally in the cell (endogenous transposons) and also those that do not occur naturally in said cell but are introduced into the cell (heterologous transposons) by means of genetic engineering methods, such as transformation of the cell, for example. Changing the expression of genes by means of transposons is known to the person skilled in the art. An overview of the use of endogenous and heterologous transposons as tools in plant biotechnology is presented in Ramachandran and Sundaresan (2001, Plant Physiology and Biochemistry 39, 234-252).

T-DNA insertion mutagenesis is based on the fact that certain sections (T-DNA) of Ti plasmids from Agrobacterium can integrate into the genome of vegetable cells. The place of integration in the vegetable chromosome is not defined, but can take place at any point. If the T-DNA integrates into a part of the chromosome or near a part of the chromosome, which constitutes a gene function, then this can lead to a reduction in the gene expression and thus also to a change in the activity of a protein encoded by the gene concerned.

Here, the sequences inserted into the genome (in particular transposons or T-DNA) are distinguished by the fact that they contain sequences, which lead to a reduction of expression or activity of an LSF-2 gene.

In a further preferred embodiment, the present invention relates to plant cells or plants according to the invention where the foreign nucleic acid molecule coding for a LSF-2 protein is selected from the group consisting of:
a) nucleic acid molecules, characterized in that they code for a LSF-2 protein originating from Arabidopsis, preferably from Arabidopsis thaliana,
b) nucleic acid molecules, characterized in that they code for a LSF-2 protein having the amino acid sequence shown in SEQ ID NO 2 or a sequence complementary thereto,
c) nucleic acid molecules coding for a protein whose sequence is at least 60%, preferably at least 80%, with preference at least 90%, especially preferably at least 95% and most preferably at least 98% identical to the amino acid sequence given under SEQ ID NO 2 or a sequence complementary thereto,
d) nucleic acid molecules comprising a nucleic acid sequence shown in SEQ ID NO 1 or a sequence complementary thereto,
e) nucleic acid molecules which are at least 70%, preferably at least 80%, with preference at least 90%, especially preferably at least 95% and most preferably at least 98% identical to the nucleic acid sequences described under b) or d),
f) nucleic acid molecules, coding for a LSF-2 protein, where the nucleic acid sequences coding for the LSF-2 protein are linked to regulatory elements, preferably with regulatory elements being promoter sequences which initiate transcription in plant cells,
g) nucleic acid molecules according to f), where the promoters are tissue-specific promoters, particularly preferably promoters which initiate the transcription specifically in tuber, fruit or seed cells of plants,
h) nucleic acid molecules which hybridize under stringent conditions with at least one strand of the nucleic acid sequences described under b) or d),
i) nucleic acid molecules whose nucleotide sequence differs from the sequence of the nucleic acid molecules mentioned under b) or d) owing to the degeneration of the genetic code; and
j) nucleic acid molecules which are fragments, allelic variants and/or derivatives of the nucleic acid molecules mentioned under a), b) or d,
k) nucleic acid molecules encoding a protein derived, or
l) a nucleic acid molecule according to b) having substitution, deletion or addition of base pairs and encoding a protein having the activity of a LSF-2 protein.

With the help of the sequence information of nucleic acid molecules encoding LSF-2 proteins described by the invention, it is possible for the person skilled in the art to isolate sequences homologous to the gene encoding the Arabidopsis LSF-2 from other plant species, preferably from starch-storing plants, preferably from plant species of the genus Oryza, in particular Oryza sativa or from Triticum sp. or from maize species. This can be carried out, for example, with the help of conventional methods such as the examination of cDNA or genomic libraries with suitable hybridisation samples. The person skilled in the art knows that homologous sequences can also be isolated with the help of (degenerated) oligonucleotides and the use of PCR-based methods.

Within the framework of the present invention, the term “hybridising” means hybridisation under conventional hybridisation conditions, preferably under stringent conditions such as, for example, are described in Sambrock et al., Molecular Cloning, A Laboratory Manual, 3rd edition (2001) Cold Spring Harbor Laboratory Press, Cold Spring Harbor, N.Y. ISBN: 0879695773, Ausubel et al., Short Protocols in Molecular Biology, John Wiley & Sons; 5th edition (2002), ISBN: 0471250929). Particularly preferably, “hybridising” means hybridisation under the following conditions:

### Hybridisation Buffer:

2×SSC; 10×Denhardt solution (Ficoll 400+PEG+BSA; Ratio 1:1:1); 0.1% SDS; 5 mM EDTA; 50 mM Na2HPO4; 250 μg/ml herring sperm DNA; 50 μg/ml tRNA; or 25 M sodium phosphate buffer pH 7.2; 1 mM EDTA; 7% SDS

### Hybridisation Temperature:

### T=65 to 68° C.

Wash buffer: 0.1×SSC; 0.1% SDS
Wash temperature: T=65 to 68° C.

In principle, nucleic acid molecules, which hybridise with the nucleic acid molecules according to the invention, can originate from any plant species, which encodes an appropriate protein. Preferably they originate from starch-storing plants, more preferably from species of the (systematic) family Poacea, particularly preferably from wheat, maize or rice. Nucleic acid molecules, which hybridise with the molecules according to the invention, can, for example, be isolated from genomic or from cDNA libraries. The identification and isolation of nucleic acid molecules of this type can be carried out using the nucleic acid molecules according to the invention or parts of these molecules or the reverse complements of these molecules, e.g. by means of hybridisation according to standard methods (see, for example, Sambrook et al., Molecular Cloning, A Laboratory Manual, 3rd edition (2001) Cold Spring Harbor Laboratory Press, Cold Spring Harbor, N.Y. ISBN: 0879695773, Ausubel et al., Short Protocols in Molecular Biology, John Wiley & Sons; 5th edition (2002), ISBN: 0471250929) or by amplification using PCR. Nucleic acid molecules, which exactly or essentially have the nucleotide sequence specified under SEQ ID NO: 1 or parts of these sequences, can be used as hybridisation samples. The fragments used as hybridisation samples can also be synthetic fragments or oligonucleotides, which have been manufactured using established synthesising techniques and the sequence of which corresponds essentially with that of a nucleic acid molecule according to the invention. If genes have been identified and isolated, which hybridise with the nucleic acid sequences according to the invention, a determination of this sequence and an analysis of the characteristics of the proteins encoded by this sequence should be carried out in order to establish whether an LSF-2 protein is involved. Homology comparisons on the level of the nucleic acid or amino acid sequence and a determination of the enzymatic activity are particularly suitable for this purpose. The activity of an LSF-2 protein can be determined as indicated elsewhere in this application.

The molecules hybridising with the nucleic acid molecules according to the invention particularly include fragments, derivatives and allelic variants of the nucleic acid molecules according to the invention, which encode an LSF-2 protein from plants, preferably from starch-storing plants, preferably from wheat, maize or rice plants. In conjunction with the present invention, the term “derivative” means that the sequences of these molecules differ at one or more positions from the sequences of the nucleic acid molecules described above and have a high degree of identity with these sequences. Here, the deviation from the nucleic acid molecules described above can have come about, for example, due to deletion, addition, substitution, insertion or recombination.

In the context of the present invention, the term “identity” means a sequence identity over the entire length of the coding region of a nucleic acid molecule or the entire length of an amino acid sequence coding for a protein of at least 60%, in particular in identity of at least 70%, preferably of at least 80%, particularly preferably of at least 90% and especially preferably of at least 95% and most preferably at least 98%. In the context of the present invention, the term “identity” is to be understood as meaning the number of identical amino acids/nucleotides (identity) with other proteins/nucleic acids, expressed in percent. Preferably, the identity with respect to a protein having the activity of a LSF-2 is determined by comparison with the amino acid sequence given under SEQ ID NO 2 and the identity with respect to a nucleic acid molecule coding for a protein having the activity of a LSF-2 protein is determined by comparison with the nucleic acid sequence given under SEQ ID NO 1 with other proteins/nucleic acids with the aid of computer programs. If sequences to be compared with one another are of different lengths, the identity is to be determined by determining the identity in percent of the number of amino acids which the shorter sequence shares with the longer sequence. Preferably, the identity is determined using the known and publicly available computer program ClustalW (Thompson et al., Nucleic Acids Research 22 (1994), 4673-4680). ClustalW is made publicly available by Julie Thompson (Thompson@EMBL-Heidelberg.DE) and Toby Gibson (Gibson@EMBL-Heidelberg.DE), European Molecular Biology Laboratory, Meyerhofstrasse 1, D 69117 Heidelberg, Germany. ClustalW can also be down-loaded from various internet pages, inter alia from IGBMC (Institut de Génétique et de Biologie Moléculaire et Cellulaire, B.P.163, 67404 Illkirch Cedex, France; ftp://ftp-igbmc.u-strasbg.fr/pub/) and from EBI (ftp://ftp.ebi.ac.uk/pub/software/) and all mirrored internet pages of the EBI (European Bioinformatics Institute, Wellcome Trust Genome Campus, Hinxton, Cambridge CB10 1SD, UK).

The examination of databases, such as are made available, for example, by EMBL (http://www.ebi.ac.uk/Tools/index.htm) or NCBI (National Center for Biotechnology Information, http://www.ncbi.nlm.nih.gov/), can also be used for identifying homologous sequences, which encode an LSF-2 protein. In this case, one or more sequences are specified as a so-called query. This query sequence is then compared by means of statistical computer programs with sequences, which are contained in the selected databases. Such database queries (e.g. blast or fasta searches) are known to the person skilled in the art and can be carried out by various providers. If such a database query is carried out, e.g. at the NCBI (National Center for Biotechnology Information, http://www.ncbi.nlm.nih.gov/), then the standard settings, which are specified for the particular comparison inquiry, should be used. For protein sequence comparisons (blastp), these are the following settings: Limit entrez=not activated; Filter=low complexity activated; Expect value=10; word size=3; Matrix=BLOSUM62; Gap costs: Existence=11, Extension=1.

For nucleic acid sequence comparisons (blastn), the following parameters must be set: Limit entrez=not activated; Filter=low complexity activated; Expect value=10; word size=11.

With such a database search, the sequences described in the present invention can be used as a query sequence in order to identify further nucleic acid molecules, which encode an LSF-2 protein, or further LSF-2 proteins. With the help of the described methods, it is also possible to identify and/or isolate nucleic acid molecules according to the invention, which hybridise with the sequence specified under SEQ ID NO: 1 and which encode an LSF-2 protein.

Identity furthermore means that there is a functional and/or structural equivalence between the nucleic acid molecules in question or the proteins encoded by them. The nucleic acid molecules which are homologous to the molecules described above and represent derivatives of these molecules are generally variations of these molecules which represent modifications having the same biological function. They may be either naturally occurring variations, for example sequences from other species, or mutations, where these mutations may have occurred in a natural manner or were introduced by targeted mutagenesis. Furthermore, the variations may be synthetically produced sequences. The allelic variants may be either naturally occurring variants or synthetically produced variants or variants generated by recombinant DNA techniques. Special forms of derivatives are, for example, nucleic acid molecules which differ from the nucleic acid molecules described in the context of the present invention owing to the degeneration of the genetic code.

For expressing nucleic acid molecules according to the invention, which encode an LSF-2 protein, these are preferably linked with regulatory DNA sequences. One example for regulatory elements are sequences which guarantee transcription in plant cells. In particular, these include promoters. In general, any promoter that is active in plant cells is eligible for expression.

At the same time, the promoter can be chosen so that expression takes place constitutively or only in a certain tissue, at a certain stage of the plant development or at a time determined by external influences. The promoter can be homologous or heterologous both with respect to the plant and with respect to the nucleic acid molecule under the conditions set out above for “heterologous” promoters. Suitable promoters are, for example, the promoter of the 35S RNA of the cauliflower mosaic virus, the rice actin promoter (Mc Elroy et al. 1990, The Plant Cell, Vol. 2, 163-171) and the ubiquitin promoter from maize for constitutive expression, the patatin promoter B33 (Rocha-Sosa et al., EMBO J. 8 (1989), 23-29) for tuber-specific expression in potatoes or a promoter, which only ensures expression in photosynthetically active tissues, e.g. the ST-LS1 promoter (Stockhaus et al., Proc. Natl. Acad. Sci. USA 84 (1987), 7943-7947; Stockhaus et al., EMBO J. 8 (1989), 2445-2451) or, for endosperm-specific expression of the HMG promoter from wheat, the USP promoter, the phaseolin promoter, promoters of zein genes from maize (Pedersen et al., Cell 29 (1982), 1015-1026; Quatroccio et al., Plant Mol. Biol. 15 (1990), 81-93), glutelin promoter (Leisy et al., Plant Mol. Biol. 14 (1990), 41-50; Zheng et al., Plant J. 4 (1993), 357-366; Yoshihara et al., FEBS Lett. 383 (1996), 213-218) or shrunken-1 promoter (Werr et al., EMBO J. 4 (1985), 1373-1380). However, promoters can also be used, which are only activated at a time determined by external influences (see for example WO 9307279). Promoters of heat-shock proteins, which allow simple induction, can be of particular interest here. Furthermore, seed-specific promoters can be used, such as the USP promoter from Vicia faba, which guarantees seed-specific expression in Vicia faba and other plants (Fiedler et al., Plant Mol. Biol. 22 (1993), 669-679; Baumlein et al., Mol. Gen. Genet. 225 (1991), 459-467). Promoters driving expression in the endosperm include the TAPR60 promoter (Kovalchuk et al. (2009). Plant Mol Biol 71:81-98), the HMW glutenin promoter (Thomas and Flavell, The Plant Cell Online December 1990 vol. 2 no. 12 1171-1180) and the PG5a promoter (U.S. Pat. No. 7,700,835).

Intron sequences can also be present between the promoter and the coding region. Such intron sequences can lead to stability of expression and to increased expression in plants (Callis et al., 1987, Genes Devel. 1, 1183-1200; Luehrsen, and Walbot, 1991, Mol. Gen. Genet. 225, 81-93; Rethmeier, et al., 1997; Plant Journal. 12(4):895-899; Rose and Beliakoff, 2000, Plant Physiol. 122 (2), 535-542; Vasil et al., 1989, Plant Physiol. 91, 1575-1579; XU et al., 2003, Science in China Series C Vol. 46 No. 6, 561-569). Suitable intron sequences are, for example, the first intron of the sh1 gene from maize, the first intron of the polyubiquitin gene 1 from maize, the first intron of the EPSPS gene from rice or one of the two first introns of the PAT1 gene from Arabidopsis.

Within the scope of the present disclosure, use may also be made of other regulatory sequences. Non-limiting examples of such regulatory sequences include transcriptional activators (“enhancers”), for instance the translation activator of the tobacco mosaic virus (TMV) described in Application WO 87/07644, or of the tobacco etch virus (TEV) described by Carrington & Freed 1990, J. Virol. 64: 1590-1597, or introns as described elsewhere in this application. Other suitable regulatory sequences include 5′ UTRs. As used herein, a 5′UTR, also referred to as leader sequence, is a particular region of a messenger RNA (mRNA) located between the transcription start site and the start codon of the coding region. It is involved in mRNA stability and translation efficiency. For example, the 5′ untranslated leader of a petunia chlorophyll at binding protein gene downstream of the 35S transcription start site can be utilized to augment steady-state levels of reporter gene expression (Harpster et al., 1988, Mol Gen Genet. 212(1):182-90). WO95/006742 describes the use of 5′ non-translated leader sequences derived from genes coding for heat shock proteins to increase transgene expression.

A further regulatory element may be a transcription termination or polyadenylation sequence operable in a plant cell, which serves to add a poly-A tail to the transcript. As a transcription termination or polyadenylation sequence, use may be made of any corresponding sequence of bacterial origin, such as for example the nos terminator of Agrobacterium tumefaciens, of viral origin, such as for example the CaMV 35S terminator, or of plant origin, such as for example a histone terminator as described in published Patent Application EP 0 633 317 A1.

Surprisingly, it has been found that plant cells according to the invention and plants according to the invention synthesise a modified starch in comparison with starch of corresponding wild type plant cells or wild type plants that have not been genetically modified.

The plant cells according to the invention and plants according to the invention synthesise a modified starch, which is altered in its physico-chemical characteristics, in particular the starch phosphate content or the phosphate distribution, in comparison with the synthesised starch in wild type plant cells or plants, so that the resulting starch is better suited for special applications.

As no enzymes have previously been described, which exclusively dephosphorylate starch at the C3-position, it has also previously not been possible to further increase the starch phosphate content specifically at the C3-position. Such an additional increase is now possible through the reduction of expression of an LSF-2 protein by the genetic modification of plants or plant cells as described herein.

Therefore, the present invention also includes plant cells and plants according to the invention, which synthesise a modified starch in comparison with corresponding wild type plant cells and wild type plants that have not been genetically modified.

In conjunction with the present invention, the term “modified starch” should be understood to mean that the starch exhibits changed physico-chemical characteristics in comparison to unmodified starch, which is obtainable from corresponding wild type plant cells or wild type plants.

In one embodiment, plant cells or plants of the invention synthesize a modified starch, characterized in that it has an increased amount of [total] starch phosphate in comparison to starch isolated from corresponding non-genetically modified wildtype plant cells or plants.

The [total] starch phosphate content of starch synthesized by the plant cells or plants of the invention may be increased by at least 50%, at least 60%, at least 65%, at least 68%, at least 70%, at least 72% or at least 75% in comparison to starch isolated from corresponding non-genetically modified wildtype plant cells or plants.

In an additional embodiment of the present invention, plant cells or plants according to the invention synthesize a starch, which contains a high content of starch phosphate at the C3-position and/or an altered phosphate distribution in comparison to starch that has been isolated from corresponding non-genetically modified wildtype plant cells and wild type plants. In other words, said plant cells or plants have an increased amount of starch phosphate bound in the C-3 position of the glucose molecules in comparison to starch isolated from corresponding non-genetically modified wildtype plant cells.

In conjunction with the current invention, the term “phosphate distribution” or “phosphate ratio” should be understood to mean the proportion of starch phosphate bonded to a glucose molecule in the, C-3 position, or C-6 position, with respect to the total starch phosphate content in the starch.

In an additional embodiment of the present invention, plant cells or plants according to the invention synthesise a starch, which exhibits an altered ratio of C-3 phosphate to C-6 phosphate in comparison to starch from wild type plants that have not been genetically modified. In particular, the modified starch is characterized in that the ratio of starch phosphate bound in the C-3 position to C-6 position of the glucose molecules is increased in comparison to the ratio of phosphate bound in the C-3 position to C-6 position of the glucose molecules in starch isolated from corresponding non-genetically modified wildtype plant cells or plants. In a specific embodiment of the invention plant cells or plants of the invention synthesize a starch, wherein the ratio of starch phosphate bound in the C-3 position to C-6 position of the glucose molecules is between 0.40-0.90 preferably 0.45-0.85 more preferably 0.50-0.80 more preferably 0.55-0.75 or most preferably 0.60-0.70.

In conjunction with the present invention, the term “ratio of C-3 phosphate to C-6 phosphate” should be understood to mean the amount of starch phosphate, of which starch phosphate bonded to starch in the C-3 position or C-6 position, respectively, contributes to the sum of the starch phosphate bonded to the starch in the C-3 position and C-6 position (C-3 position+C-6 position).

The phosphate bound in the C-3 position of the glucose molecules in the starch synthesized by plant cells or plants of the invention amounts to at least 25%, preferably at least 30%, more preferably at least 33%, even more preferably at least 35% or particularly preferred at least 37% of the total starch phosphate content.

The phosphate bound in the C-3 position of the glucose molecules in the starch synthesized by plant cells or plants of the invention amounts to at most 60%, preferably at most 58%, more preferably at most 55%, even more preferably at most 53% or particularly preferred at most 50% of the total starch phosphate content.

The phosphate bound in the C-3 position of the glucose molecules in the starch synthesized by plant cells or plants of the invention amounts to between 25%-60%, preferably between 30%-58%, more preferably between 33%-55%, even more preferably between 35%-53% or particularly between 37%-50% of the total starch phosphate content.

The phosphate bound in the C-3 position of the glucose molecules in the starch synthesized by plant cells or plants of the invention amounts to at least 0.60, preferably at least 0.65, more preferably at least 0.70, even more preferred at least 0.75, most preferred at least 0.80 or particularly preferred at least 0.85 nmol phosphate per 1 glucose equivalent. The glucose equivalent assigns to each glucose molecule being part of a glucan, e.g. starch or maltooligosaccharides the molecular mass a single glucose molecules has (180.16 g/mol).

In the context of the present invention, the starch of the invention preferably concerns starch isolated from starch storing parts of plants, grain starch or leaf starch.

In conjunction with the present invention, the term “starch-storing parts” is to be understood to mean such parts of a plant in which starch is stored as a deposit for surviving for longer periods. Preferred starch-storing plant parts are, for example, tubers, storage roots and grains, particularly preferred are grains containing an endosperm, especially particularly preferred are grains containing an endosperm of maize or wheat plants.

Different methods of determining the amount of starch phosphate are described. Preferably, the method of determining the amount of starch phosphate described by Ritte et al. (2000, Starch/Starke 52, 179-185) can be used. Particularly preferably, the determination of the amount of starch phosphate by means of 31P-NMR is carried out according to the method described by Kasemusuwan and Jane (1996, Cereal Chemistry 73, 702-707).

Furthermore, an object of the invention is genetically modified plants, which comprise or consist of plant cells according to the invention. These types of plants can be produced from plant cells according to the invention by regeneration.

In principle, the plants according to the invention can be plants of any plant species, i.e. both monocotyledonous and dicotyledonous plants. Preferably they are crop plants, i.e. plants, which are cultivated by man for the purposes of food production or for technical, in particular industrial purposes.

In a further embodiment, the plant according to the invention is a starch-storing plant. In an additional embodiment, the present invention relates to starch-storing plants according to the invention of the (systematic) family Poaceae. These are preferably rice, maize or wheat plants.

In conjunction with the present invention, the term “starch-storing plants” means all plants with plant parts, which contain a storage starch, such as, for example, maize, rice, wheat, triticale, rye, oats, barley, cassaya, potato, sago, mung bean, pea or sorghum.

In conjunction with the present invention, the term “potato plant” or “potato” means the plant species of the genus Solanum, particularly tuber-producing species of the genus Solanum, and in particular Solanum tuberosum.

In conjunction with the present invention, the term “wheat plant” means plant species of the genus Triticum or plants resulting from crosses with plants of the genus Triticum, particularly plant species of the genus Triticum or plants resulting from crosses with plants of the genus Triticum, which are used in agriculture for commercial purposes, and particularly preferably Triticum aestivum or Triticum durum. Plants obtained from such a cross include triticale plants.

In conjunction with the present invention, the term “rice plant” means plant species of the genus Oryza, particularly Oryza sativa, preferably japonica, indica or javanica rice, whether soil, water, upland, rainfed shallow, deep water, floating or irrigated rice.

In conjunction with the present invention, the term “maize plant” means plant species of the genus Zea, particularly plant species of the genus Zea, which are used in agriculture for commercial purposes, particularly preferably Zea mays.

The present invention also relates to propagation material of plants according to the invention containing a plant cell according to the invention.

Here, the term “propagation material” includes those constituents of the plant that are suitable for producing offspring by vegetative or sexual means. Cuttings, callus cultures, rhizomes or tubers, for example, are suitable for vegetative propagation. Other propagation material includes, for example, fruits, seeds, seedlings, protoplasts, cell cultures, etc. Preferably, the propagation material is tubers and particularly preferably grains, which contain endosperms.

In a further embodiment, the present invention relates to harvestable plant parts of plants according to the invention such as fruits, storage roots, roots, blooms, buds, shoots or stems, preferably seeds, grains or tubers, wherein these harvestable parts contain plant cells according to the invention.

Furthermore, the present invention also relates to a method for the manufacture of a genetically modified plant, such as a plant according to the invention, comprising a) genetically modifying a plant cell, whereby the genetic modification leads to the reduction of the activity of an LSF-2 protein in comparison with corresponding wild type plant cells that have not been genetically modified; b) regenerating a plant from the plant cell of a).

Optionally the method for the manufacture of a genetically modified plant comprises a further step c), wherein further plants are produced using the plants obtained in step b).

The genetic modification introduced into the plant cell according to Step a) can basically be any type of genetic modification, which leads a reduction in the activity of an LSF-2 protein. Suitable molecules to be introduced in line with said genetic modification as well as techniques to effect modifications are described elsewhere in this application.

The regeneration of the plants according to Step (b) can be carried out using methods known to the person skilled in the art (e.g. described in “Plant Cell Culture Protocols”, 1999, edt. by R. D. Hall, Humana Press, ISBN 0-89603-549-2).

The production of further plants according to Step (c) of the method according to the invention can be carried out, for example, by vegetative propagation (for example using cuttings, tubers or by means of callus culture and regeneration of whole plants) or by sexual propagation. Here, sexual propagation preferably takes place under controlled conditions, i.e. selected plants with particular characteristics are crossed and propagated with one another. In this case, the selection is preferably carried out in such a way that further plants, which are obtained in accordance with Step c), exhibit the genetic modification, which was introduced in Step a).

In a further embodiment of the method for the manufacture of a genetically modified plant according to the invention, the genetic modification consists in the introduction of a foreign nucleic acid molecule according to the invention into the genome of the plant cell, wherein the presence or the expression of said foreign nucleic acid molecule leads to reduced activity of an LSF-2 protein in the cell.

Specific embodiments of the foreign nucleic acid molecule to be used in the method for the manufacture of a genetically modified plant according to the invention are disclosed above in connection with the plant cell or plant of the invention and equally applicable to this aspect of the invention.

The present invention also relates to the plants obtainable or obtained by the method according to the invention.

Surprisingly, it has been found that starch isolated from plant cells according to the invention and plants according to the invention, which have a reduced activity of an LSF-2 protein, synthesize a modified starch.

In particular, the increased quantities of starch phosphate in starches according to the invention provide the starches with surprising and advantageous properties. Starches according to the invention have an increased proportion of loaded groups due to the increased proportion of starch phosphate, which considerably affect the functional properties. Starch that contains loaded functional groups is particularly usable in the paper industry, where it is utilised for paper coating. Paper, which is coated with loaded molecules that also exhibit good adhesive properties, is particularly suitable for absorbing pigments, such as dye, printing inks, etc., for example.

Accordingly, in one aspect, the present invention relates to modified starches obtainable or obtained from plant cells according to the invention or plants according to the invention, from harvestable plant parts according to the invention or from a plant obtainable or obtained by a method according to the invention.

Naturally, the characteristics of the starch as described for the starch produced by the plant cells or plants of the invention equally apply to the starch according to the present embodiment of the invention.

In a further embodiment, the present invention relates to modified starch according to the invention, isolated from starch-storing plants, preferably from starch-storing plants of the (systematic) family Poaceae, particularly preferably from maize, rice or wheat plants.

Furthermore the present invention relates to a method for the manufacture of a modified starch including the step of extracting the starch from a plant cell according to the invention or from a plant according to the invention, from propagation material according to the invention of such a plant from harvestable plant parts according to the invention of such a plant and/or from plants obtainable or obtained by a method for producing a genetically modified plant according to the invention, preferably from starch-storing parts according to the invention of such a plant. Preferably, such a method also includes the step of harvesting the cultivated plants or plant parts and/or the propagation material of these plants before the extraction of the starch and, further, particularly preferably the step of cultivating plants according to the invention before harvesting.

Methods for extracting starches from plants or from starch-storing parts of plants are known to the person skilled in the art. Furthermore, methods for extracting starch from different starch-storing plants are described, e.g. in Starch: Chemistry and Technology (Publisher: Whistler, BeMiller and Paschall (1994), 2nd Edition, Academic Press Inc. London Ltd; ISBN 0-12-746270-8; see e.g. Chapter XII, Page 412-468: Maize and Sorghum Starches: Manufacture; by Watson; Chapter XIII, Page 469-479: Tapioca, Arrowroot and Sago Starches: Manufacture; by Corbishley and Miller; Chapter XIV, Page 479-490: Potato starch: Manufacture and Uses; by Mitch; Chapter XV, Page 491 to 506: Wheat starch: Manufacture, Modification and Uses; by Knight and Oson; and Chapter XVI, Page 507 to 528: Rice starch: Manufacture and Uses; by Rohmer and Klem; Maize starch: Eckhoff et al., Cereal Chem. 73 (1996), 54-57, the extraction of maize starch on an industrial scale is generally achieved by so-called “wet milling”.). Devices, which are in common use in methods for extracting starch from plant material are separators, decanters, hydrocyclones, spray dryers and fluid bed dryers.

In conjunction with the present invention, the term “starch-storing parts” is to be understood to mean such parts of a plant in which, in contrast to transitory leaf starch, starch is stored as a deposit for surviving for longer periods. Preferred starch-storing plant parts are, for example, tubers, storage roots and grains, particularly preferred are grains containing an endosperm, especially particularly preferred are grains containing an endosperm of maize or wheat plants.

Modified starch obtainable or obtained by a method according to the invention for manufacturing modified starch is also the subject matter of the present invention.

In a further embodiment of the present invention, the modified starch according to the invention is native starch.

In conjunction with the present invention, the term “native starch” means that the starch is isolated from plants according to the invention, harvestable plant plants according to the invention, starch-storing parts according to the invention or propagation material of plants according to the invention by methods known to the person skilled in the art.

Furthermore, the use of plant cells according to the invention or plants according to the invention for manufacturing a modified starch are the subject matter of the present invention.

The person skilled in the art knows that the characteristics of starch can be changed by thermal, chemical, enzymatic or mechanical derivation, for example, to obtain derived starch. Derived starches are particularly suitable for different applications in the foodstuffs and/or non-foodstuffs sector. The starches according to the invention are better suited to be an initial substance for the manufacture of derived starches than for conventional starches, since they exhibit a higher proportion of reactive functional groups due to the higher starch phosphate content.

The present invention therefore also relates to the manufacture of a derived starch, wherein modified starch according to the invention is derived subsequent to isolation of modified starch according to the invention from plant cells or plants according to the invention.

In conjunction with the present invention, the term “derived starch” is to be understood to mean a modified starch according to the invention, the characteristics of which have been changed after isolation from vegetable cells with the help of chemical, enzymatic, thermal or mechanical methods.

In a further embodiment of the present invention, the derived starch according to the invention is starch that has been treated with heat and/or acid.

In a further embodiment, the derived starches are starch ethers, in particular starch alkyl ethers, O-allyl ethers, hydroxylalkyl ethers, O-carboxylmethyl ethers, nitrogen-containing starch ethers, phosphate-containing starch ethers or sulphur-containing starch ethers.

In a further embodiment, the derived starches are cross-linked starches.

In a further embodiment, the derived starches are starch graft polymers.

In a further embodiment, the derived starches are oxidised starches.

In a further embodiment, the derived starches are starch esters, in particular starch esters, which have been introduced into the starch using organic acids. Particularly preferably these are phosphate, nitrate, sulphate, xanthate, acetate or citrate starches.

The derived starches according to the invention are suitable for different applications in the pharmaceutical industry and in the foodstuffs and/or non-foodstuffs sector. Methods for manufacturing derived starches according to the invention are known to the person skilled in the art and are adequately described in the general literature. An overview on the manufacture of derived starches can be found, for example, in Orthoefer (in Corn, Chemistry and Technology, 1987, eds. Watson and Ramstad, Chapter 16, 479-499).

Derived starch obtainable by the method according to the invention for manufacturing a derived starch is also the subject matter of the present invention.

Furthermore, the use of modified starches according to the invention for manufacturing derived starch is the subject matter of the present invention.

Starch-storing parts of plants are often processed into flours. Examples of parts of plants from which flours are produced, for example, are tubers of potato plants and grains of cereal plants. For the manufacture of flours from cereal plants, the endosperm-containing grains of these plants are ground and strained. Starch is a main constituent of the endosperm. In the case of other plants, which do not contain endosperm, and which contain other starch-storing parts instead such as tubers or roots, for example, flour is frequently produced by mincing, drying, and subsequently grinding the storing organs concerned. The starch of the endosperm or contained within starch-storing parts of plants is a fundamental part of the flour, which is produced from those plant parts, respectively. The characteristics of flours are therefore affected by the starch present in the respective flour. Plant cells according to the invention and plants according to the invention synthesise a modified starch in comparison with wild type plant cells and wild type plants that have not been genetically modified. Flours produced from plant cells according to the invention, plants according to the invention, propagation material according to the invention, or harvestable parts according to the invention, therefore exhibit modified properties. The properties of flours can also be affected by mixing starch with flours or by mixing flours with different properties.

Therefore, an additional object of the invention relates to flours, comprising or containing a starch according to the invention.

In conjunction with the present invention, the term “flour” is to be understood to mean a powder obtained by grinding plant parts. Plant parts are possibly dried before grinding, and minced and/or strained after grinding.

A further subject of the present invention relates to flours, which are produced from plant cells according to the invention, plants according to the invention, from starch-storing parts of plants according to the invention, from propagation material according to the invention, or from harvestable plant parts according to the invention. Preferred starch-storing parts of plants according to the invention are tubers, storage roots, and grains containing an endosperm. Tubers preferably come from potato plants, and grains preferably come from plants of the (systematic) family Poaceae, while grains particularly preferably come from maize or wheat plants.

Flours according to the invention are characterised in that they contain starch, which exhibits a modified phosphate content and/or a modified phosphate distribution. Flours comprising starch with an increased amount of starch phosphate show an increased water binding capacity. This is desirable in the processing of flours in the foodstuffs industry for many applications, and in particular in the manufacture of baked goods, for example.

A further object of the present invention is a method for the manufacture of flours, including the step of grinding plant cells according to the invention, plants according to the invention, parts of plants according to the invention, starch-storing parts of plants according to the invention, propagation material according to the invention, harvestable material according to the invention or respective plants or parts thereof obtainable or obtained by a method for producing a genetically modified plants of the invention.

Flours can be produced by grinding starch-storing parts of plants according to the invention. Methods for the manufacture of flours are known to the person skilled in the art. A method for the manufacture of flours preferably includes the step of harvesting the cultivated plants or plant parts and/or the propagation material or the starch-storing parts of these plants before grinding, and particularly preferably includes the additional step of cultivating plants according to the invention before harvesting.

In conjunction with the present invention, the term “parts of plants” should be understood to mean all parts of the plants that, as constituents, constitute a complete plant in their entirety. Parts of plants are scions, leaves, rhizomes, roots, knobs, tubers, pods, seeds, or grains.

In a further embodiment of the present invention, the method for the manufacture of flours includes processing plants according to the invention, starch-storing plants according to the invention, propagation material according to the invention, or harvestable material according to the invention before grinding.

In this case, processing can be heat treatment and/or drying, for example. Heat treatment followed by a drying of the heat-treated material is used in the manufacture of flours from storage roots or tubers such as potato tubers, for example, before grinding. The mincing of plants according to the invention, starch-storing parts of plants according to the invention, propagation material according to the invention, or harvestable material according to the invention before grinding can also represent processing in the sense of the present invention. The removal of other plant tissue before grinding, such as e.g. grain husks, also represents processing before grinding in the sense of the present invention.

In a further embodiment of the present invention, the method for the manufacture of flours includes processing the ground product.

In this case, the ground product can be strained after grinding, for example, in order to produce various types of flours, for example.

A further subject of the present invention is the use of genetically modified plant cells according to the invention or plants according to the invention for the manufacture of flours.

In another aspect, the present invention relates to a chimeric gene comprising (a) a heterologous promoter, which initiates transcription in plant cells, and (b) a nucleic acid sequence, selected from the group consisting of: i. nucleic acid sequences encoding an LSF-2 protein or a part thereof ii. nucleic acid sequences which upon expression of at least one antisense RNA, effects a reduction in the expression of at least one endogenous gene, which encodes an LSF-2 protein, iii. which by means of a co-suppression effect lead to the reduction in the expression of at least one endogenous gene, which encodes an LSF-2 protein, iv. which encode at least one ribozyme, which splits specific transcripts of at least one endogenous gene, which encodes an LSF-2 protein; or v. which simultaneously express at least one antisense RNA and at least one sense RNA, wherein the said antisense RNA and the said sense RNA form a double-stranded RNA molecule, which effects a reduction in the expression of at least one endogenous gene, which encodes an LSF-2 protein.

Specific embodiments of the nucleic acids have been described herein already in connection with genetically modified plant cells or plants of the invention. These specific embodiments are also applicable to specific embodiments of the chimeric gene according to the invention.

A chimeric gene is an artificial gene constructed by operably linking fragments of unrelated genes or other nucleic acid sequences. In other words “chimeric gene” denotes a gene which is not naturally found in a plant species or refers to any gene in which the promoter or one or more other regulatory regions of the gene are not associated in nature with a part or all of the transcribed nucleic acid, i.e. are heterologous with respect to the transcribed nucleic acid.

The term “heterologous” refers to the relationship between two or more nucleic acid or protein sequences that are derived from different sources. For example, a promoter is heterologous with respect to an operably linked nucleic acid sequence, such as a coding sequence, if such a combination is not naturally found in nature. In addition, a particular sequence may be “heterologous” with respect to a cell or organism into which it is inserted (i.e. does not naturally occur in that particular cell or organism). For example, the chimeric gene disclosed herein is a heterologous nucleic acid.

Nucleic acids can be DNA or RNA, single- or double-stranded. Nucleic acids can be synthesized chemically or produced by biological expression in vitro or even in vivo. Nucleic acids can be chemically synthesized using appropriately protected ribonucleoside phosphoramidites and a conventional DNA/RNA synthesizer. Suppliers of RNA synthesis reagents are Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK).

In connection with the chimeric gene of the present disclosure, DNA includes cDNA and genomic DNA.

In one embodiment of the chimeric gene of the invention, said nucleic acid encoding an LSF-2 protein comprises the nucleotide sequence of SEQ ID NO: 1 or a nucleotide sequence at least 80%, at least 90%, at least 95% or at least 98% identical to SEQ ID NO: 1.

In a further embodiment of the chimeric gene of the invention, said nucleic acid comprises a nucleotide sequence encoding a LSF-2 protein comprising the amino acid sequence as shown under SEQ ID NO 2, or a nucleic acid sequence encoding a protein having at least 80%, at least 90%, at least 95% or at least 98% identical to SEQ ID NO: 2.

A further embodiment of chimeric genes according to the invention comprises vectors, in particular plasmids, cosmids, viruses, bacteriophages and other common vectors in genetic engineering, which contain the chimeric genes according to the invention described above.

A further subject of the present invention is a host cell, in particular a prokaryotic or eukaryotic cell, which is genetically modified with a recombinant nucleic acid molecule according to the invention and/or with a vector according to the invention, as well as cells, which originate from host cells of this type and which contain the chimeric gene according to the invention.

In a further embodiment, the invention relates to host cells, particularly prokaryotic or eukaryotic cells, which were transformed with a nucleic acid molecule according to the invention or a vector according to the invention, as well as host cells, which originate from host cells of this type and which contain the chimeric gene or vectors according to the invention.

The host cells are preferably microorganisms. Within the framework of the present application, these are understood to mean all bacteria and all protista (e.g. fungi, in particular yeast and algae), as defined, for example, in Schlegel “Allgemeine Mikrobiologie” (Georg Thieme Verlag (1985), 1-2).

The host cells can be bacteria (e.g. E. coli, bacteria of the genus Agrobacterium in particular Agrobacterium tumefaciens or Agrobacterium rhizogenes) or fungus cells (e.g. yeast, in particular S. cerevisiae, Agaricus, in particular Agaricus bisporus, Aspergillus, Trichoderma), as well as plant or animal cells. Here, the term “transforms” means that the cells according to the invention are genetically modified with a chimeric gene according to the invention inasmuch as they contain at least one chimeric gene according to the invention in addition to their natural genome. This can be freely present in the cell, possibly as a self-replicating molecule, or it can be stably integrated in the genome of the host cell.

It is preferred if the host cells according to the invention are plant cells as also described elsewhere in this application. In principle, these can be plant cells from all species already described herein.

Also a subject of the present invention are compositions containing a chimeric gene according to the invention, or a vector according to the invention. Preferred are compositions according to the invention containing a chimeric gene according to the invention, or a vector according to the invention and a host cell. It is particularly preferred that the host cell is a plant cell, and especially preferred that it is a cell of a maize, rice or wheat plant.

A further aspect of compositions according to the invention relates to compositions, which can be used for producing host cells according to the invention, preferably for producing plant cells according to the invention. Preferably this concerns a composition containing nucleic acid sequences coding an LSF-2 protein such as that represented in SEQ ID NO: 1, a chimeric gene according to the invention or a vector according to the invention and a biolistic carrier, which is suitable for the introduction of nucleic acid molecules into a host cell. Preferred biolistic carriers are particles of tungsten, gold or synthetic materials.

In another aspect, the invention relates to the use of a chimeric gene according to the invention, a vector according to the invention, a host cell, such as a plant cell, according to the invention or a composition according to the invention for the production of a genetically modified plant cell or plant, preferably for the production of a genetically modified plant according to the invention.

A further object of the invention is the use of a chimeric gene of the invention for preparing a vector according to the invention, a host cell, a plant cell or plant according to the invention or for the production of a composition according to the invention. Furthermore the use of a chimeric gene according to the invention for the production of a modified starch is an object of the invention.

The invention also relates to a protein having the activity of an LSF-2 protein, i.e. it de-phosphorylates the phosphate residue bound in the C-3 position of the glucose molecules in the starch.

### Description of the Sequences

SEQ ID NO:1: Nucleic acid molecule encoding a LSF-2 protein from Arabidopsis thaliana.

SEQ ID NO:2: Amino acid sequence for a LSF-2 protein from Arabidopsis thaliana. The amino acid shown can be derived by translation of SEQ ID NO 1.

Primers used for genotyping homozygous mutant
plants
- T-DNA left border primers
SEQ ID NO: 3:
LBb1 Sail; GCCTTTTCAGAAATGGATAAATAGCCTTGCTTCC
SEQ ID NO: 4:
LBb1 Salk; GCGTGGACCGCTTGCTGCAACT
SEQ ID NO: 5:
DS3-2 (for GT10871); CCGGTATATCCCGTTTTCG
- Lsf-2-1 (Sail 595 F04)
SEQ ID NO: 6:
SAIL_595 F04 LP; ATATTGCGGTGCAACTTTACG
SEQ ID NO: 7:
SAIL_595 F04 RP; CTGAGCATTTATCAGTTGGGG
- Lsf-2-2 (GT10871)
SEQ ID NO: 8:
At3g10940_fw1; TGTGATTGGAAGCAAGAGCT
SEQ ID NO: 9:
At3g10940_re1; CCGAACACGTTCTTGAATCAAC
- Sex4-3 (Salk_102567)
SEQ ID NO: 10:
Salk_102567 LP; AAGCTGATGCGTAATGAATCG
SEQ ID NO: 11:
Salk_102567 RP; GAAATCCCCAAACATCCTCAC
Primers used for qRT-PCR
- PP2A houskeeping gene (At1g13320)
SEQ ID NO: 12:
PP2A_F01; CTCTTACCTGCGGTAATAACTG
SEQ ID NO: 13:
PP2A_R01; CATGGCCGTATCATGTTCTC
- LSF2 gene (At3g10940)
SEQ ID NO: 14:
At3g10940_F01; GACATGAATCTTAACACGGCT
SEQ ID NO: 15:
At3g10940_R01; ATCATATGTTGCACCACGG
Primers used for recombinant cloning
- Subcellular localization (LSF2-GFP)
SEQ ID NO: 16:
LSF2pGFP2 fw (KpnI site added);
GGGGTACCATGAGTGTGATTGGAAGC
SEQ ID NO: 17:
LSF2pGFP2 rev (KpnI site added);
GGGGTACCGGTTCCACGGAGGGCC
- Construction of LSF2prom: GUS fusion gene
SEQ ID NO: 18:
LSF2prom fw; GATTGCATTATTGATTTGTTGCTCTTGTAG
SEQ ID NO: 19:
LSF2prom rev; CGTTCTCTATCTCTCGTTCTTCACCTG
- Cloning of recombinant LSF-2 wt
SEQ ID NO: 20:
LSF2 full length cDNA fw; ATGAGTGTGATTGGAAGCAAGAGC
SEQ ID NO: 21:
LSF2 full length cDNA rev; TCAGGTTCCACGGAGGGC
- Cloning of Δ65-LSF2
SEQ ID NO: 22:
Δ65-LSF2_fw; TTTCATATGAACAAAATGGAGGATTACAATACAGC
SEQ ID NO: 23:
Δ65-LSF2_rev; AAACTCGAGTCATCAGGTTCCACGGAGGGCC
- Cloning of Δ78-LSF2
SEQ ID NO: 24:
Δ78-LSF2_fw;
TTTCATATGATGAGAAGCCCTTATGAATATCATCATG
SEQ ID NO: 25:
Δ78-LSF2_rev; AAACTCGAGTCATCAGGTTCCACGGAGGGCC
- Cloning of LSF2ΔCT
SEQ ID NO: 26:
LSF2-CT fw, GAATGATCCCTGAAAAGAGCCCTTTG
SEQ ID NO: 27:
LSF2-CT rev; CAAAGGGCTCTTTTCAGGGATCATTC
- Cloning of LSF2 C/S
SEQ ID NO: 28:
LSF2 C193S fw;
GGTAAAGGAAGAGTCTATGTGCATTCTTCAGCCGGATTGG
SEQ ID NO: 29:
LSF2 C193S rev;
CCAATCCGGCTGAAGAATGCACATAGACTCTTCCTTTACC

SEQ ID NO:30: Peptide sequence from a LSF-2 protein identified in Arabidopsis thaliana; DFDPLSLR

SEQ ID NO:31: Peptide sequence from a LSF-2 protein identified in Arabidopsis thaliana; DFDPLSLR

SEQ ID NO:32: Peptide sequence from a LSF-2 protein identified in Arabidopsis thaliana; AVSSLEWAVSEGK.

SEQ ID NO:33: Peptide sequence from a LSF-2 protein identified in Arabidopsis thaliana; DELIVGSQPQKPEDIDHLK

SEQ ID NO:34: Peptide sequence from a LSF-2 protein identified in Arabidopsis thaliana; KLIQER

### DESCRIPTION OF THE FIGURES

FIG. 1: LSF2 protein structure, heterologous expression and sub-cellular localization.

A. Schematic representation of the domain topography of SEX4, LSF2 and LSF1. The chloroplast targeting peptide is in light grey (cTP), the dual specificity phosphatase (DSP) domain in striped, the carbohydrate binding module (CBM) in dotted, the PDZ-like domain in dark grey, and the C-terminal domain in black. The active site of the proteins is denoted with a black line. The lengths of the proteins are also indicated.

B. Surface view of the LSF2 homology model based on the SEX4 crystal structure (Vander Kooi et al., 2010), showing the predicted integrated architecture between the DSP (right part) and C-terminal domains (very left part).

C. Ribbon diagram of the predicted LSF2 structure (B). Elements of secondary structure are numbered consecutively from the N to C termini.

D. The C-terminal domain is essential for soluble expression of LSF2. Coomassie stained SDS page showing purification of Δ65LSF2 protein and Δ65LSF2 CT which lacks the C-terminal 35 residues. UI, uninduced cells, I, cells induced with IPTG, P, pellet of insoluble protein, S, soluble protein.

E. Subcellular localization of transiently expressed LSF2-GFP fusion protein in Arabidopsis wildtype protoplasts. Green fluorescence of the control GFP protein is found in the cytosol, while LSF2— GFP fusion protein is located in the chloroplast. Transmission images of the same cells are also shown. Green fluorescence, transmission and chlorophyll pictures were merged to show the accurate localization of GFP fluorescence in the chloroplast. Note how the untransformed protoplast only shows chlorophyll autofluorescence (lower chloroplast). Scale bar, 20 μm.

FIG. 2: Structural elements and sequence similarities between SEX4 and LSF2.

A. Sequence alignment of Arabidopsis LSF2 and SEX4. Secondary structures of SEX4, some of which are also predicted for LSF-2, are displayed as ovals (α-helices) and arrows (β-sheets) using the following patterns: 1) striped, SEX4-specific elements, including the CBM; 2) black, LSF-2-specific elements; 3) light gray, common α-helix in the cTP. The predicted cTP cleavage site is marked with a box; 4) intermediate gray, α-helices and 3-sheets in the DSP domain common to both proteins; 5) dark gray, α-helices in the C-terminal domain common to both proteins.

B. Percent similarity (in black) and identity (in red) between Arabidopsis SF2 and SEX4 for the full length proteins (left), for the dual specificity phosphatase domain (DSP), and carboxy-terminal domain (CT), as indicated.

C. The C-terminal domain is essential for soluble expression of LSF2. Coomassie stained SDS page showing the purification of LSF2 protein (32 kDa) and LSF2ΔCT protein (28 kDA) which lacks the C-terminal 35 residues. UI, uninduced cells; I, cells induced with IPTG; P, pellet of insoluble protein; S, soluble protein; E, eluted fraction.

FIG. 3: Temporal and spatial expression pattern of the LSF2 gene.

A-G. GUS reporter gene expression in transgenic Arabidopsis plants carrying the β-glucuronidase gene fused downstream of the LSF2 promoter. (A) Seven-day-old seedlings. After 6 h, Staining was strongest in cotyledons, the vasculature, the lower part of the hypocotyl and the root-shoot junction. (B) 7-day-old etiolated seedlings. Staining was observed only in the vasculature. (C and D) Roots of light grown 7-day-old seedlings (as in (A)). Staining was detected in the central cylinder and the root tip and the lateral root primordia. (E and F) Floral organs and developing siliques. Strong staining was observed in the sepal vasculature, the stamen and the distal part of the style. (G) A decrease in staining was observed in cotyledons of light grown seedlings after 72 h of dark treatment (samples were stained for 24 h).

H. Expression levels of LSF2 in different organs at different developmental stages. Data were retrieved from the public eFP browser microarray dataset ‘Developmental map’ (http://www.bar.utoronto.ca/efp/cgi-bin/efpWeb.cgi).

I. Comparison of expression profiles of SEX4 and LSF2 genes over a diurnal cycle (12 h dark/12 h light). Expression values were normalized to the median of all eleven time points for each gene. Data used in this analysis are from Smith et al. (2004) and were retrieved from the NASC website (http://www.nasc.nott.ac.uk/).

J. Numbers of peptides identified in different tissue proteomes of Arabidopsis (http://fgczatproteome.unizh.ch/). Overall, the number of identified peptides is representative of protein abundance.

FIG. 4: The intron-exon structure of the homologous genes LSF2, LSF1 and SEX4. Exons (cylinders), introns (black) lines, not to scale) and the 5′ and 3′ untranslated regions (blue lines, not to scale) are shown, Coloured exons encode the DSP domain and the CBM, as indicated. Dashed lines indicate conserved intron positions. The locations of the T-DNA and transposon insertions within the LSF2 gene are shown (510 and 1016 bp downstream of the ATG start codon for Isf2-2 and Isf2-1, respectively). Line identifiers are given in red. The insertion site sequences are shown. The sequence is given above the insert, with the gene in lower case and the T-DNA or the Ds transposon in uppercase. The length of the intervening sequence not derived from either the T-DNA, Da transposon or the gene is shown in parenthesis.

FIG. 5: LSF2 is a starch-binding phosphoglucan phosphatase specific for C3-bound phosphate esters in starch.

A. Specific activity of SEX4, LSF2 and active site mutant LSF2 C/S with the artificial substrate p-NPP (p-nitrophenyl phosphate) at their optimal pH (6.5). Error bars indicate mean±SE (n=3; p value<0.05).

B. Phosphate release measured by the malachite green assay using SEX4, LSF2 or active site mutant LSF2 C/S against solubilized amylopectin (left) and purified sex4 phospho-oligosaccharides (right) at their respective optimal pH (6.5). The amounts of the two phosphatases (SEX4 and LSF2) used in the assay were adjusted to equal hydrolytic activity on p-NPP, whilst the amounts of the two substrates were normalized to similar amounts of phosphate. Note the different scales on the Y axes. Error bars indicate mean±SE (n=3). Similar results were obtained using Δ78-LSF2 recombinant protein lacking the N-terminal 78 residues (corresponding to the chloroplast transit peptide).

C. Hydrolysis of C6- and C3-phosphate esters in native starch granules. Purified phosphate-free starch granules from GWD-deficient Arabidopsis sex1-3 mutants (Yu et al., 2001) were pre-labeled with ³³P at either C6- or C3-positions and incubated with SEX4, LSF2, or active site mutant LSF2 C/S recombinant proteins. Phosphate release over time was linear and is expressed relative to the total ³³P incorporated into starch. Reaction time was 5 min. Each value is the mean±SE of 4 replicate samples.

D. Binding of LSF2 to potato amylose free (waxy) starch in vitro. SEX4, Δ78-LSF2 and AP (alkaline phosphatase from calf intestine) proteins were incubated with starch for 30 min at 20° C. The starch was pelleted by centrifugation. Proteins in the supernatant (S), in the pellet wash (W) and bound to the pellet (P) were visualized by SDS-PAGE and silver-staining.

FIG. 6: LSF2-mediated hydrolysis of C6- and C3-phosphate esters at native starch granules.

Purified phosphate-free starch granules from the GWD-deficient Arabidopsis mutant sex1-3 were pre-labeled with ³³P at either C6- or C3-positions and incubated with 5 μg of LSF2 recombinant protein for 2 h. At intervals during the 2-h time course, the released ³³P was determined. After 15 min LSF2 dephosphorylated exclusively C3-phospho esters, as expected. However, after 2 h LSF2 also released small amounts of phosphate from the C6-position.

FIG. 7: SDS-PAGE of proteins binding to starch granules.

Arabidopsis proteins were incubated with amylase free potato starch and bound proteins were eluted with SDS (Binding). Proteins binding to isolated Arabidopsis starch were extracted (Internal). The boxes indicate the regions of the gels that were subjected to in-gel tryptic digestion and analyzed by LC-MS/MS.

FIG. 8: Phenotypic characterization of Isf2 mutant alleles.

A. Quantitative RT (Reverse Transcriptase)-PCR analysis of LSF2 gene expression in leaves of 4-week-old plants. Transcript level for each line was normalized to the expression of the PP2A housekeeping gene (At1g13320). Transcript levels in Isf2 plants are given relative to the respective wild-type plants.

B. Release of 33P from isolated starch granules by crude extracts of wild-type and Isf2 leaves. Purified phosphate free starch granules from GWD-deficient Arabidopsis mutants sex1-3 were pre-labeled with ³³P at either C6- or C3-positions and were then incubated with desalted leaf extracts. Phosphate release over time was linear under these conditions and is expressed relative to the phosphate released by the corresponding wild-type extracts. Each value is the mean±SE of 4 replicate samples.

C. Leaf starch content at the end of the day and the end of the night (as indicated) in the wild types Col-0 and Ler-0 and in the Isf2-1, Isf2-2 mutants. Each value is the mean±SE of eight replicate samples. FW, fresh weight.

D. Starch-bound phosphate content in Isf2-1 and Isf2-2 mutant alleles and their respective wild types. The phosphate content of starch purified from leaves of 4-week-old plants harvested at the end of the light period is shown as grey bars. The amylopectin content for the same starch preparations was determined to be 92.6%±0.2% for Col-0, 91.4%±0.1% for Isf2-1, 88.7%±0.4% for Ler, and 88.4%±0.5% for Isf2-2. Amylopectin-bound phosphate (black bars) was calculated assuming all the phosphate is bound to amylopectin. Each value is the mean±SE of four replicate samples.

FIG. 9: Hydrolysis of C6- and C3-phosphate esters from starch granules by extracts of the wild type, Isf2 and sex4.

Purified phosphate-free starch granules from GWD-deficient Arabidopsis sex1-3 mutants were prelabeled with ³³P at either C6- or C3-positions, and were then incubated with desalted extracts from whole rosettes of wild type Col-0, sex4, Isf2 plants harvested at the end of the light period. Phosphate release over time was linear under these conditions and was expressed relative to the phosphate released by wild-type extracts. Each value is the mean±SE of 4 replicate samples.

FIG. 10: Impact of the Isf2 mutation on starch metabolism and plant growth.

A. Photographs of wild type Col-0 and Isf2, sex4 mutants harvested at the end of the day (top) and at the end of the night (bottom) after 4 weeks of growth. To visualize starch content, chlorophyll was cleared from the plants in 80% (v/v) ethanol and stained for starch with iodine solution. Representative plants were selected to show the reduced growth rate of mutant.

Fresh weight average values are given above (g, n=6). Scale bar, 1 cm.

B. Leaf starch content at the end of the day (grey bars) and at the end of the night (black bars) in the wild type Col-0 and Isf2, sex4 mutants. Each value is the mean±SE of nine replicate samples (p value<0.05). FW, fresh weight.

C. Phospho-oligosaccharide content at the end of the day (grey bars) and at the end of the night (black bars) in the wild type Col-0 and Isf2, sex4—mutants. Each value is the mean±SE of nine replicate samples (p value<0.05). FW, fresh weight.

FIG. 11: The Isf2 mutation causes elevated C3-bound phosphate levels.

31P NMR 1D spectra of hydrolyzed starch of wild type, Isf2, sex4, sex1 and pwd plants harvested at the end of the light period recorded with between 9216 and 16384 transients at 303 K, pH 6.0. Peak areas are proportional to the relative amount of glucan-bound phosphate and are given as a percentage on top of each peak. Chemical shifts are referenced to external H₃PO₄ (85%).

### MATERIAL AND GENERAL METHODS

### Plant Materials and Growth Conditions

Plants for metabolite measurements were grown in a controlled environment chamber (Percival AR-95L, CLF Plant Climatics GmbH, Wertingen, Germany) in a 12-h light/12-h dark cycle with a constant temperature of 22° C., 65% relative humidity, and a uniform illumination of 150 μmol photons m⁻² s⁻¹. Plants used for the preparation of leaf starch granules were grown in a climate chamber (Weiss Umwelttechnik GmbH, Reiskirchen-Lindenstruth, Germany) with 16-h light/8-h dark regime with a constant temperature of 21° C. and 60% relative humidity. Light intensity was between 120-140 μmol photons m⁻²s⁻¹. To promote uniform germination, imbibed seeds were stratified for 3 days at 4° C. in the dark.

The following Arabidopsis thaliana T-DNA insertion mutants were used in this study: sex4-3 (Salk_{—}102567; Niittyla et al., 2006, J. Biol. Chem. 281, 11815-11818), sex1-3 (Yu et al., 2001, Plant Cell 13, 1907-1918), pwd (SALK_{—}110814, Kötting et al., 2005, Plant Physiol. 137, 242-252), Isf2-1 (Sail_{—}595_F04, this work), Isf2-2 (GT10871, this work). Arabidopsis ecotype Columbia (Col-0) was used in all experiments, except for Isf2-2 which was in a Ler ecotype background.

### LSF2 Subcellular Localization

To localize LSF2, its coding sequence was amplified from a full-length cDNA obtained from the Riken Bioresource Center (stock pda16983) and cloned in frame with the N-terminus of GFP in the vector pGFP2 (Haseloff and Amos, 1995, Trends Genet. 11, 328-329). The LSF2-GFP fusion protein was transiently expressed in isolated Arabidopsis mesophyllprotoplasts as described previously (Fitzpatrick and Keegstra, 2001, Plant J. 27, 59-65). GFP fluorescence and chlorophyll autofluorescence were monitored using a confocal laser scanning microscope (TCS-NT; Leica Microsystems, Heerbrugg, Switzerland) with excitation windows of 507 to 520 nm and 620 to 700 nm, respectively. TCS-NT software version 1.6.587 was used for image acquisition and processing.

### Quantitative Reverse Transcription PCR

Total RNA was extracted from leaves using an RNeasy Mini Kit (Qiagen, Hombrechtikon, Switzerland). Following DNase-I treatment, 1 μg of total RNA of each sample was used to produce cDNA using oligo dT primer (18-mer) and SuperScript® III First-Strand Synthesis System (Invitrogen, Basel, Switzerland). qPCR was performed using the SYBR Green Supermix (Eurogentec, S. A. Ougrée, Belgium) with an iCycler (Applied Biosystems, Carlsbad, Calif., USA). Reactions were run in triplicate with three different cDNA preparations, and the iQ5 Optical System Software (Applied Biosystems) was used to determine the threshold cycle (Ct) when fluorescence significantly increased above background. Gene-specific transcripts were normalized to PP2A gene (At1g13320) and quantified by the ΔCt method (Ct of gene of interest—Ct of PP2A gene). Real-time SYBR-green dissociation curves showed one species of amplicon for each primer combination.

### Construction of LSF2pro::GUS Fusion Gene and Arabidopsis Transformation

A DNA fragment corresponding to 1.5 kb of genome sequence upstream of the LSF2 start codon was amplified from Arabidopsis genomic DNA by PCR and sequenced to confirm that there was no spontaneous mutation introduced. The DNA fragment was inserted into the binary vector pMDC163 (Curtis and Grossniklaus, 2003, Plant Physiol. 133, 462-469) upstream of the GUS reporter gene to create a recombinant unit LSF2pro::GUS. The reporter gene fusion was introduced into wild-type Arabidopsis plants (Col-0) through Agrobacterium tumefaciens-mediated transformation using the floral dip method (Clough and Bent, 1998, Plant J. 16, 735-743). The independent transformants were selected on half-strength Murashige and Skoog media (Dufecha Biochemie, Haarlem, Netherlands) supplemented with hygromycin (50 μg ml-1) and transferred to soil after 2-3 weeks. T2 plants (i.e. progeny of transgenic generation 1) were used for the GUS staining.

### Histochemical GUS Staining and Microscopy

Seedlings were immersed in GUS staining solution (50 mM sodium phosphate buffer pH 7.0, 0.05% (w/v) X-Gluc, 1 mM K₃[Fe(CN)₆], 1 mM K₄[Fe(CN)₆], 0.05% (v/v) Triton X-100) and infiltrated under vacuum for 30 min. Staining proceeded for 4 or 16 h at 37° C. Chlorophyll was removed with 70% (v/v) EtOH and the plant tissues examined using conventional light microscopy. Images of GUS staining patterns are representative of at least three independent transgenic lines.

### Homology Modeling of LSF2

HHpred search (Soding, 2005, Bioinformatics 21, 951-960; Soding et al., 2005, Nucleic Acids Res. 33, W244-248) and InterPro domain scan (Zdobnov and Apweiler, 2001, Bioinformatics 17, 847-848) were utilized to determine which DSP structure was the best template to model LSF2. The top hits were aligned with LSF2 using PROfile Multiple Alignment with predicted Local Structure 3D (PROMALS3D) (Zdobnov and Apweiler, 2001, Bioinformatics 17, 847-848). These alignments were the inputs in alignment mode of SWISS-MODEL from Swiss PDB viewer version 8.05 (Arnold et al., 2006, Bioinformatics 22, 195-201).

Multiple homology models were inspected manually and by Anolea, Gromos, and Verify3d (Luthy et al., 1992, Nature 356, 83-85; Melo et al., 1997, Proc. Int. Conf. Intell. Syst. Mol. Biol. 5, 187-190; Christen et al., 2005, J. Comput. Chem. 26, 1719-1751). The model utilizing SEX4 (Protein Data Bank code 3 nm e) as the template was the best model.

### Phylogenetic Analysis

Genomic sequence and gene models of Arabidopsis lyrata, Arabidopsis thaliana, Ricinus communis, Vitis vinifera, Oryza sativa, Zea mays, Sorghum bicolor, Selaginella moellendorffii, Physcomitrella patens, Populus trichocarpa, Glycine max, Zea mays, Volvox carteri, Chlamydomonas reinhardtii, Chlorella variabilis, Ostreococcus tauri, Osterococcus lucimarinus, Micromonas sp. Micromonas pusilla, Mus musculus, Gallus gallus, Homo sapiens, Paramecium tetraurelia, Tetrahymena thermophila, Cyanidioschyzon merolae, Thalassiosira pseudonana, Porphyra yezoensis, Caenorhabditis elegans, Plasmodium falciparum, Guillardia theta as well as all completed bacterial genomes were mined using BLASTx and tBLASTn (cut off of e⁻⁴) with LSF1, LSF2 and SEX4 from Arabidopsis. Results were subject to reciprocal BLAST against the Arabidopsis genome and proteins with a different top hit were noted and the corresponding Arabidopsis sequences were added to the results. All protein sequences were aligned using CLUSTALx (Thompson et al., 1997, Nucleic Acids Res. 25, 4876-4882) and the alignment was imported into MacClade (Sinauer Associates, MA. USA) for refinement. All proteins of bacterial origin as well as proteins with reciprocal results other than LSF1, LSF2 and SEX4, were easily alignable within the DSP domain. However, they generally encoded additional domains not present in LSF1, LSF2 or SEX4, which severely compromised the inclusion set within DSP and, after distance analysis of the DSP domain, were confirmed to be more related to other proteins.

These were excluded from further analysis leaving only proteins from eukaryotic species, the majority from plants, green algae and metazoans. The DSP domains of remaining proteins were realigned with CLUSTALx. Ambiguously aligned characters were excluded in MacClade and any sequences of the same species that were identical after exclusion of ambiguous characters were also collapsed to a single taxon, resulting in a matrix of 65 taxa and 150 characters. Maximum likelihood (ML) phylogenies were inferred using (a) PhyML (Guindon and Gascuel, 2003, Syst. Biol. 52, 696-704) with the Dayoff substitution matrix and eight categories of substitution rates and (b) RAxML7.04 software (Stamakis, 2006, Bioinformatics 22, 2688-2690) using GTR+GAMMA model of evolution. The alpha value and number of invariable sites were calculated from the datasets. The branching support was assessed using ML bootstrap analysis (PhyML with four rate categories and 100 replications, RAxML, GTR+GAMMA and 1000 replications) and Bayesian posterior probability values based on 1,000,000 generations and priors set to default using MrBayes 3.1.2 (Ronquist and Huelsenbeck, 2003, Bioinformatics 19, 1572-1574).

### Cloning, Expression and Purification of Recombinant Proteins

The full length cDNA of LSF2 was cloned into pProEXHT vector (Invitrogen, Base1, Switzerland) according to standard protocols. Additional pET28b LSF2 constructs were generated where we truncated the first 78 or 65 amino acids (pET28b Δ78-LSF2 and pET28b Δ65-LSF2, respectively) or the last 35 amino acids (pET28b LSF2ΔCT and pET28b Δ65LSF2ΔCT). pET21 Δ52-SEX4 has been previously described (Gentry et al., 2007, J. Cell Biol. 178, 477-488). A point mutation in the LSF2 gene resulting in the C193S substitution was generated with the QuickChange Site-Directed Mutagenesis kit (Agilent Technologies, Basel, Switzerland) according to the manufacturer's instructions and cloned into pProEXHta vector. Recombinant proteins were expressed with an amino- or carboxy-terminal hexahistidine tag in E. coli BL21 (DE3) CodonPlus cells (Stratagene, Basel, Switzerland). Fusion proteins were expressed and purified from soluble extracts of E. coli using Ni2+-NTA agarose affinity chromatography as described previously (Kötting et al., 2005, Plant Physiol. 137, 242-252).

### Measurement of Phosphatase Activity

Phosphatase activity of recombinant enzymes against para-nitrophenylphosphate (p-NPP, Fluka, Buchs, Switzerland), solubilized amylopectin (Sigma-Aldrich, Buchs, Switzerland), and purified phospho-oligosaccharides was measured using modifications of previously described methods (Worby et al., 2006, J. Biol. Chem. 281, 30412-30418). In all assays, the amount of SEX4 and LSF2 recombinant enzymes used was 0.05 μg and 0.2 μg, respectively. For p-NPP hydrolysis, each enzyme was incubated with 50 mM p-NPP at 37° C. in 50 μL reactions with SEX4 assay medium containing 100 mM sodium acetate, 50 mM bis-Tris, 50 mM Tris, 2 mM dithiothreitol (DTT); pH 6.5. Reactions were stopped at specific times by addition of 200 μL 250 mM NaOH. The amount of released p-NPP was quantified by measuring absorbance at 410 nm. Activity against solubilized potato amylopectin or purified phospho-oligosaccharides was determined by measuring released orthophosphate using the malachite green reagent. Phospho-oligosaccharides were isolated from extracts of sex4 mutants as previously described (Kotting et al., 2009, Plant Cell 21, 334-346). Recombinant enzymes were incubated with solubilized amylopectin (equivalent to 45 μg dry weight) or purified phospho-oligosaccharides (equivalent to 2 nmol phosphate) at 37° C. in 20 μL reactions with assay medium (see as above). Reactions were stopped with 20 μL of N-ethylmaleimide (250 mM) after the indicated incubation times. Subsequently, 80 μL of the malachite green reagent (2.5% (w/v) (NH₄)₆Mo₇O₂₄, 0.15% (w/v) malachite green in 1 M HCl) was added, and the color allowed to develop at 20° C. for 10 min. Absorbance at 660 nm was used to determine the phosphate groups released against a standard curve prepared with K₂HPO4. Assays were performed in triplicate.

### Starch Binding Assays

The starch binding capacity of recombinant LSF2 and SEX4 (Gentry et al., 2007) was determined in vitro. Calf intestine alkaline phosphatase (Fermentas, Nunningen, Switzerland) served as a non starch binding control. Each enzyme (5 μg) was incubated on a rotating wheel with pre-hydrated amylose-free potato starch (equivalent to 30 mg dry weight) at room temperature for 30 min in a final volume of 250 μL with assay medium (as above). Starch was pelleted by centrifugation and unbound proteins remained in the supernatant. The pellet was washed once in 250 μL assay medium.

Then, bound proteins were eluted by re-suspending the starch pellet in 100 μL total protein extraction buffer (40 mM Tris-HCl, pH 6.8, 5 mM MgCl₂, 4% SDS, and Complete Protease Inhibitor Cocktail (Roche, Basel, Switzerland) for 30 minutes at 37° C. The soluble fraction and the supernatant from the wash were concentrated to 100 μL in Amicon Ultra spin concentrators (Molecular weight cut-off of 10 kDa; Millipore, Zug, Switzerland). Equal volumes of the concentrated unbound fraction and the eluted bound fraction were subjected to SDS-PAGE and visualized by silver-staining. The activity of unbound proteins in the supernatant was measured against p-NPP (see Measurement of Phosphatase Activity, above), and compared to control reactions that contained no starch.

### Starch Granule Protein Analysis by Tandem Mass Spectrometry

Arabidopsis proteins were extracted from rosettes in 40 mM Tris-HCl, pH 6.8, 5 mM MgCl₂, 1 mM DTT, 1 mM phenylmethylsulfonyl fluoride. Extracted proteins (45 mg) were incubated for 4 h with 6 g of potato starch in a final volume of 50 mL at 4° C. The starch was collected by centrifugation, washed once with the same medium, and bound proteins eluted with protein extraction buffer (see above).

Arabidopsis starch with bound proteins was isolated as described previously (Ritte et al., 2000, Plant J. 21, 387-391). To extract proteins bound to the surface of the granules, the starch was incubated with the total protein extraction buffer described above for the starch binding assays. Proteins encapsulated inside the starch granules were subsequently isolated by boiling the granules in a buffer containing SDS as described by Boren et al (2004), except with omission of DTT (Boren et al., 2004). Extracted proteins were separated and visualized by Coomassie-stained SDS-PAGE. For proteomics analysis, gel slices were diced into small pieces and in gel digestion was performed as describe previously (Shevchenko et al., 1996, Anal. Chem. 68, 850-858). After digestion, peptides were dried in a speedvac and subsequently dissolved in 3% (v/v) acetonitrile 0.2% (v/v) trifluoretic acid. Peptides were then desalted using Sepak C18 Cartridges (Waters, Milford, Mass., USA), re-dried in the speedvac, and then re-dissolved in 3% (v/v) acetonitrile, 0.2% (v/v) formic acid and analyzed on a FT-ICR mass spectrometer (Thermo Fisher Scientific, Bremen, Germany) according to previously described methods (Agne et al., 2010, Plant Physiol. 153, 1016-1030).

MS/MS spectra were searched with Mascot (Matrix Science, London, UK) version 2.2.04 against the Arabidopsis TAIR10 protein database (download on Jan. 17, 2011) with a concatenated decoy database supplemented with contaminants. The search parameters were: requirement for tryptic ends, one missed cleavage allowed, mass tolerance=+/−5 ppm. Beside carbamidomethylation of cysteines as fixed modification, oxidation of methionine was included as variable modification. Peptide identification was accepted with a minimal Mascot ion score of 26 and a Mascot expectation value below 0.05 resulting in a false positive rate at peptide level below 1% for all measured samples.

### Iodine Staining

Four-week-old Arabidopsis rosettes were harvested at the end of day or end of night, and were incubated in 80% (v/v) ethanol for 12 h to remove the chlorophyll. The cleared plants were rinsed in water and stained in Lugol solution (Sigma-Aldrich, Buchs, Switzerland) for 10 min.

### Starch and Phospho-Oligosaccharide Extraction and Quantification

For starch content and phospho-oligosaccharide measurements, whole rosettes from 4-week-old Arabidopsis plants were harvested at the end of day or end of night, weighed, then snap frozen in liquid N₂. Subsequent analyses were performed as previously described (Kotting et al., 2009, Plant Cell 21, 334-346).

### GWD and PWD Protein Quantification

To extract total proteins, whole rosettes of 4-week-old Arabidopsis plants were harvested and immediately snap frozen in liquid N₂. The entire rosette was homogenized in total protein extraction buffer as described in starch binding assays section. Extracted proteins were quantified using the Pierce BCA Protein Assay Kit (Thermo Fisher Scientific, Wohlen, Switzerland). Equal amounts of protein were separated by SDS-PAGE, electroblotted onto PVDF, and probed with an antibody raised against potato GWD (Ritte et al., 2000, Plant J. 21, 387-391) or Arabidopsis PWD (Kotting et al., 2005, Plant Physiol. 137, 242-252). The GWD and PWD bands, detected by chemiluminescence using a ChemiGlow West kit (Cell Biosciences Santa Clara, Calif., USA), was quantified using gel analysis tools on ImageJ software (v1.42q; NIH, USA).

### Quantification of Total Phosphate on Starch

Starch was isolated from whole Arabidopsis rosettes as described previously (Kotting et al., 2005, Plant Physiol. 137, 242-252). Starch granules (5 mg) were acid-hydrolyzed in 50 μL 2 M HCl for 2 h at 95° C. The reaction was neutralized with 100 μL 1 M NaOH, and 50 μL was incubated with 15 units of Antarctic Phosphatase (New England Biolabs, Frankfurt am Main, Germany) for 2 h at 37° C. in a final volume of 100 μL with assay medium (see above). Released orthophosphate was determined using the malachite green reagent, as above.

Phosphate Release from ³³P Labelled Granules

Phosphate-free starch granules isolated from the Arabidopsis sex 1-3 mutant (Yu et al., 2001) were pre-phosphorylated with ³³P at the C6- or C3-position as described in (Hejazi et al., 2010). In both cases, the starch granules were phosphorylated at both locations, but the ³³P-label was only at one or the other position. Recombinant potato GWD and recombinant Arabidopsis PWD were generated as described elsewhere (Ritte et al., 2002, Proc. Natl. Acad. Sci. USA 99, 7166-7171; Kotting et al., 2005, Plant Physiol. 137, 242-252). [(3⁻³³P]-ATP was from Hartmann Analytic (Braunschweig, Germany). Recombinant SEX4, LSF2 or LSF2 C/S (50 ng in each case) was incubated in dephosphorylation medium (100 mM sodium acetate, 50 mM bis-Tris, 50 mM Tris-HCl, pH 6.5, 0.05% (v/v) Triton X-100, 1 μg/μl (w/v) BSA, and 2 mM DTT) with 4 mg ml⁻¹ starch pre-labelled at either the C6- or the C3-position (see above) in a final volume of 150 μl on a rotating wheel for 5 min at 20° C.

Crude extracts of soluble protein were produced from 4-week-old Arabidopsis plants by homogenizing whole rosettes in a medium containing 50 mM 4-(2-hydroxyethyl)-1-piperazineethanesulfonic acid (HEPES)-KOH pH 7.5, 1 mM EDTA, 5 mM DTT, 10% (v/v) glycerol and Complete Protease Inhibitor Cocktail (Roche, Rotkreuz, Switzerland). Extracts were desalted using NAP-5 Sephadex G-25 columns (GE Healthcare, Glattbrugg, Switzerland). Protein (37.5 μg) from these extracts were incubated with 0.75 mg of either C3- or C6-³³P-labelled granules at 20° C. for 20 min in reaction medium containing 50 mM HEPES-KOH, pH 7.0, 5 mM MgCl₂, 5 mM CaCl₂, 0.1% (w/v) BSA, 2 mM DTT and 0.025% (v/v) Triton X-100) at a final reaction volume of 150 μL.

Reactions were stopped by adding 50 μL of 10% (w/v) SDS, and the starch was pelleted by centrifugation. The amount of ³³P released into the medium was quantified by scintillation counting.

The starch pellet in blank reactions was also counted, and the data expressed as the percentage ³³P released into the supernatant. Phosphate release over time was linear under these conditions.

### NMR Analysis of Starch-Bound Phosphate

Starch was isolated from Arabidopsis wild-type and mutant lines as described above and 50 mg was suspended in 500 μL of medium containing 3 mM NaCl, 1 mM CaCl₂, and 60 μg of α-amylase from pig pancreas (Roche, Mannheim, Germany). The suspension was shaken vigorously at 95° C. for 5 min until the starch had gelatinized. A further 50 μg α-amylase and 450 μg amyloglucosidase from Aspergillus niger (Roche) was added, and digestion carried out at 37° C. for 12 h with shaking after which the solution was clear and non-viscous. All NMR measurements were performed on an AVANCE III 600 MHz spectrometer equipped with a QCI CryoProbe (Bruker, Fallanden, Switzerland) at 303 K. Prior to analysis, the pH was adjusted to 6.0 with 0.2 M NaOH and 5% (v/v) D₂O was added to all samples. 1D ³¹P spectra were recorded with 9200-16400 transients, a recycle delay of 3.8 s and 1H WALTZ16 decoupling (Shaka et al., 1983, J. Magn. Reson. 53, 313-340) at a field strength of 2.8 kHz.

Spectra were indirectly referenced to H₃PO₄ (85% wt solution in H₂O; AppliChem, Darmstadt, Germany) using a Ξ value of 0.404807356 (Maurer and Kalbitzer, 1996, J. Magn. Reson. Ser., B. 113, 177-178). All spectra were processed with Topspin 2.1 (Bruker). Glucose-3-phosphate (Glycoteam GmbH, Hamburg, Germany) and glucose-6-phosphate (Roche, Rotkreuz, Switzerland) were used as a reference for peak identification.

### Matrix Assisted Laser Desorption Ionization Mass Spectrometry (MALDI/MS/MS) Analysis of Phosphorylated Oligosaccharides

One milligram of wild-type digested starch (as prepared for NMR analysis) was dissolved in 25 μl of 10% (v/v) acetonitrile. Samples were spotted on the target by mixing 1 μl of matrix (40 mg ml⁻¹ 2,5-dihydroxybenzoic acid (DHB) in 50% (v/v) acetonitrile, 1% (w/v) H₃PO₄ for wild-type starch and 15 mg ml⁻¹ DHB in 30% (v/v) acetonitrile, 0.1% (v/v) trifluoroacetic acid (TFA) for the rest) with 1 μl of each sample and then analyzed by MALDI (Ultraflex II TOF/TOF equipped with a smart beam laser) in the reflector negative ion mode. The analysis range was 500-5000 m/z.

### Example 1

### LSF2 is a Chloroplastic Protein Homolog of SEX4

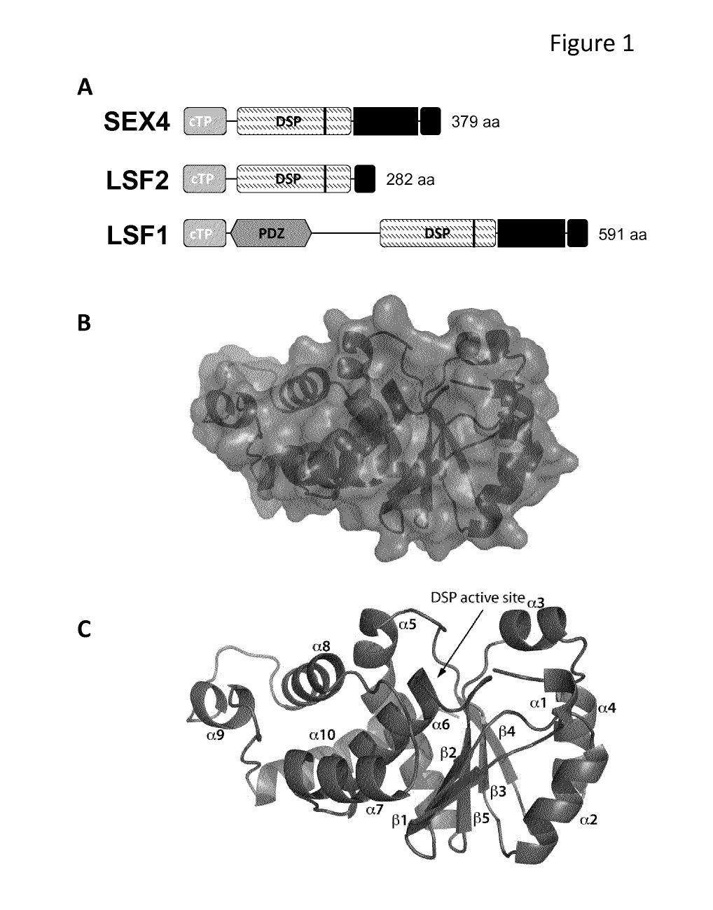

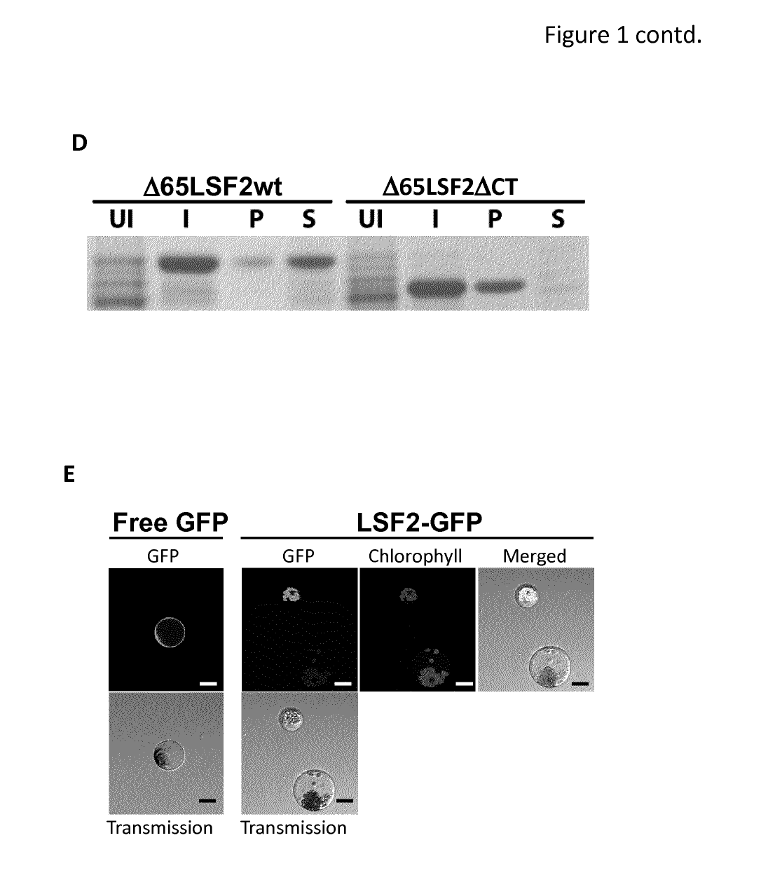

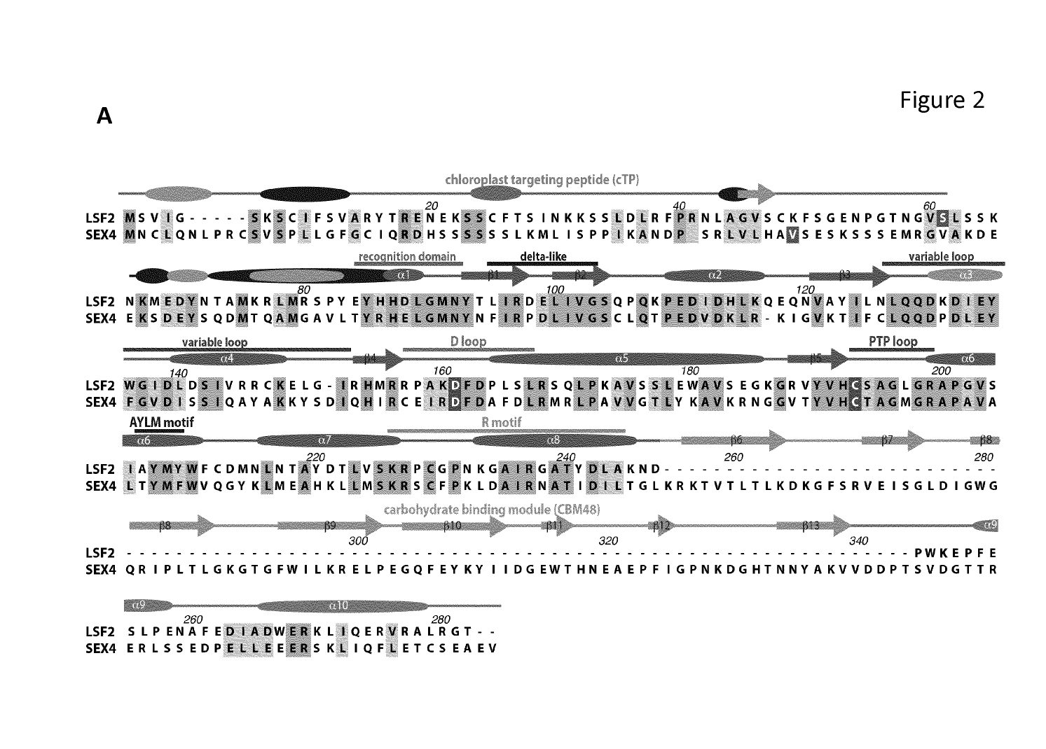

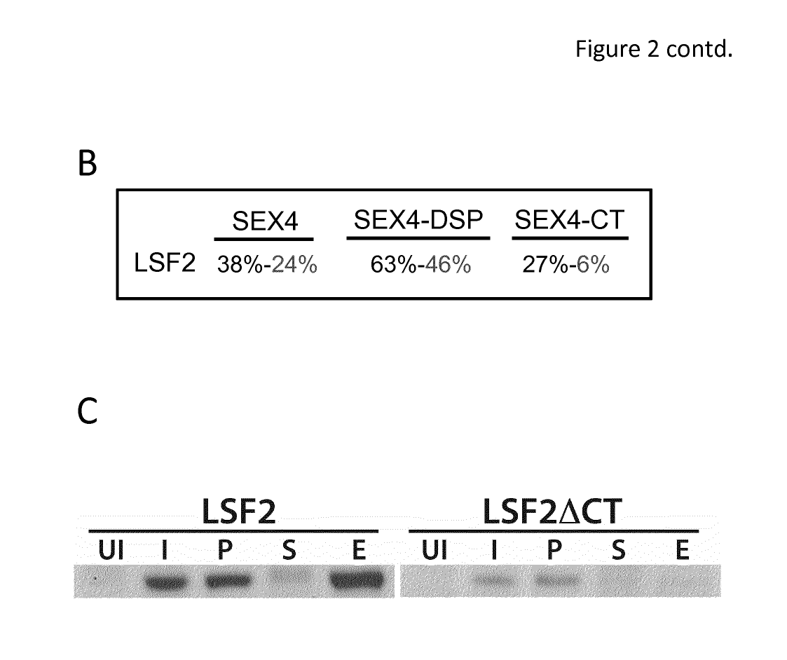

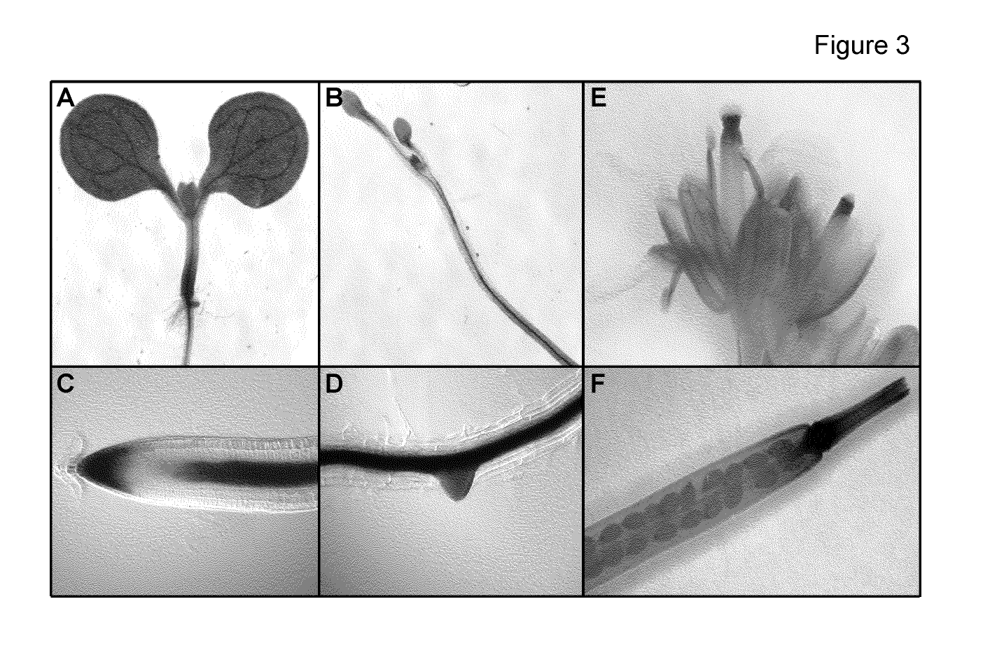

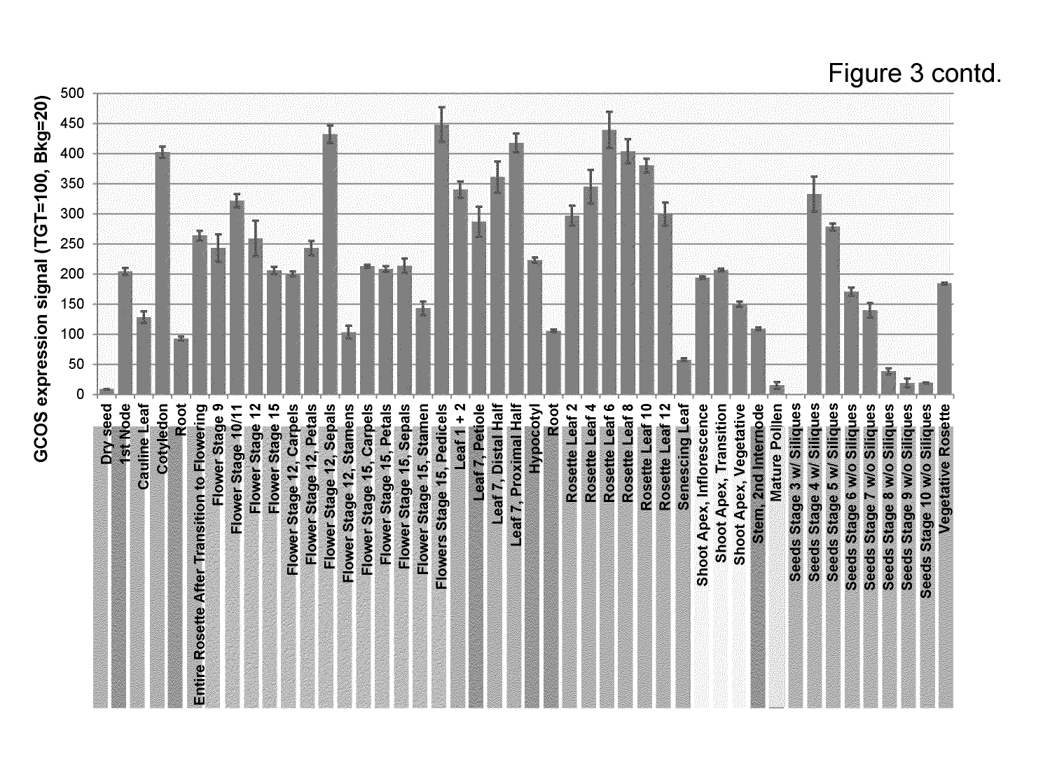

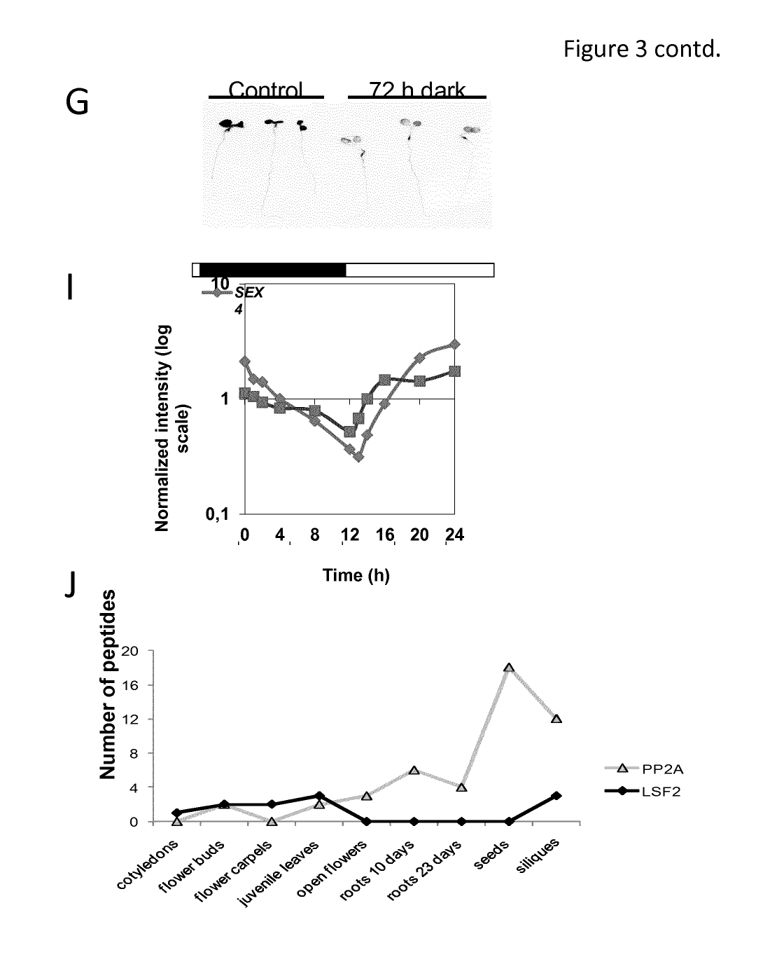

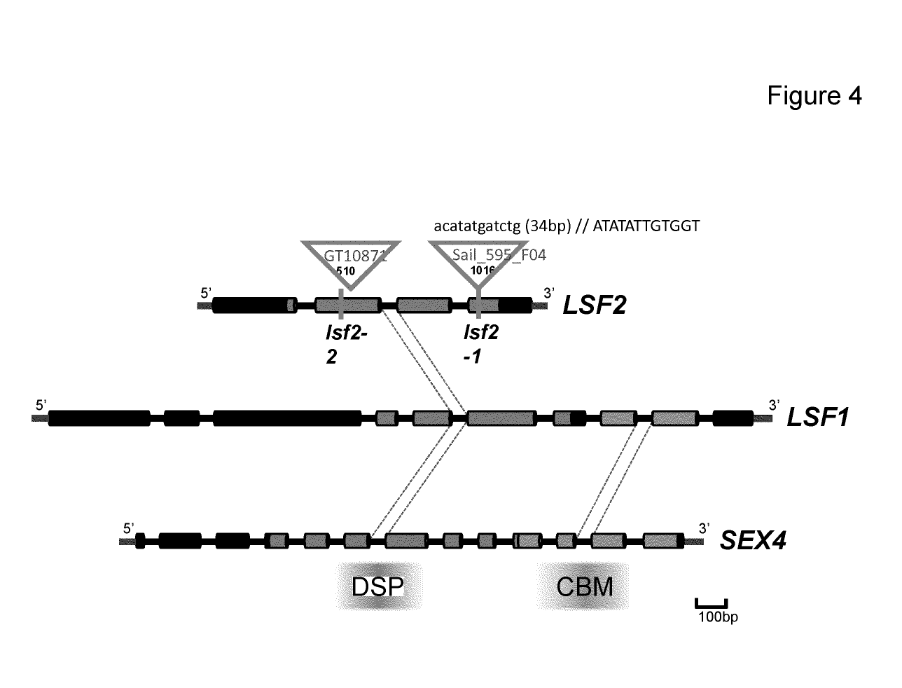

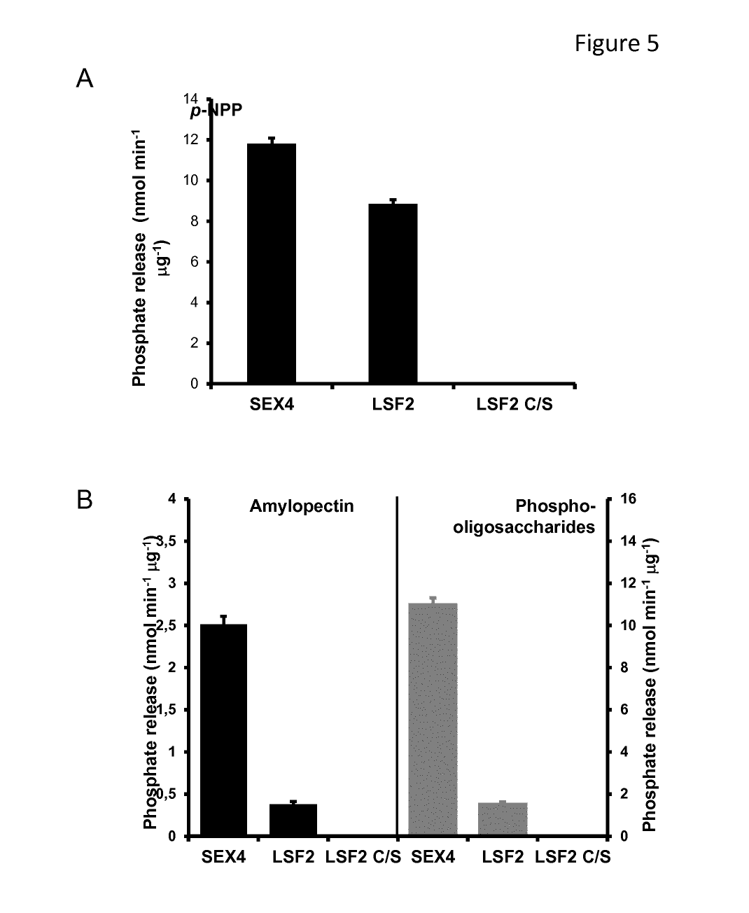

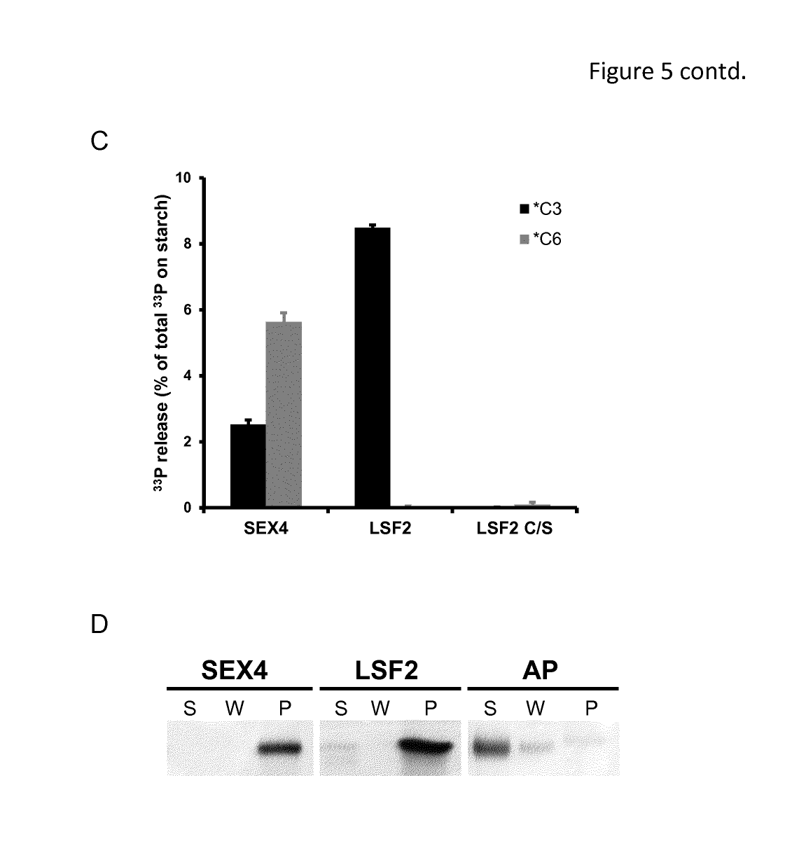

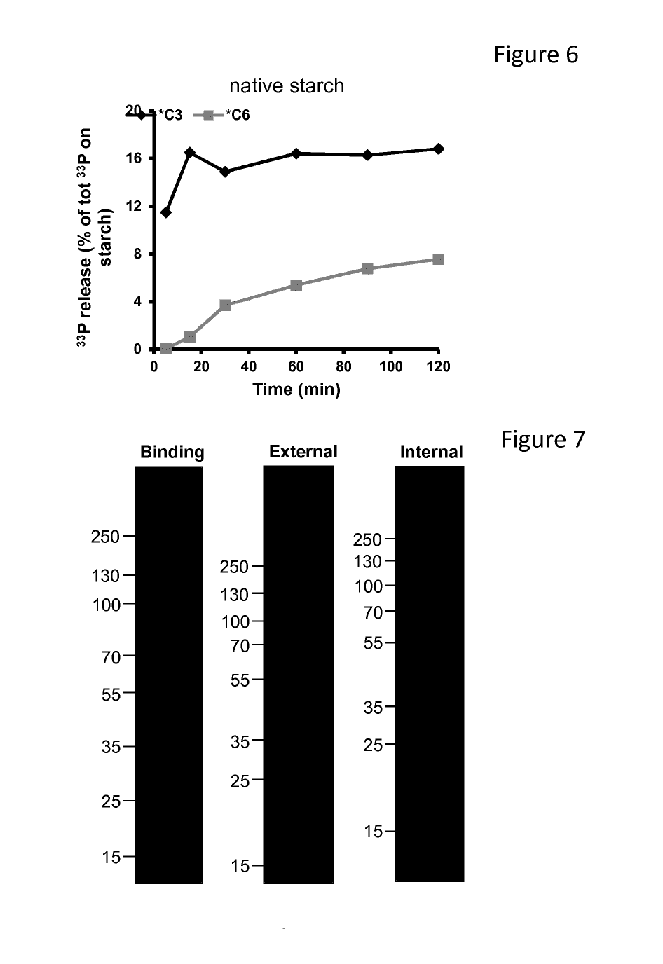

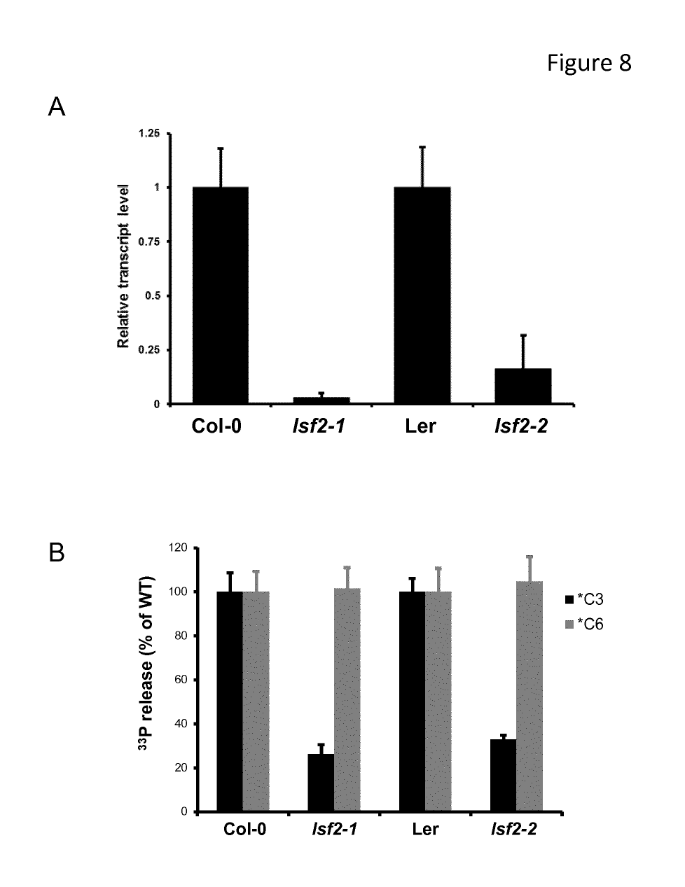

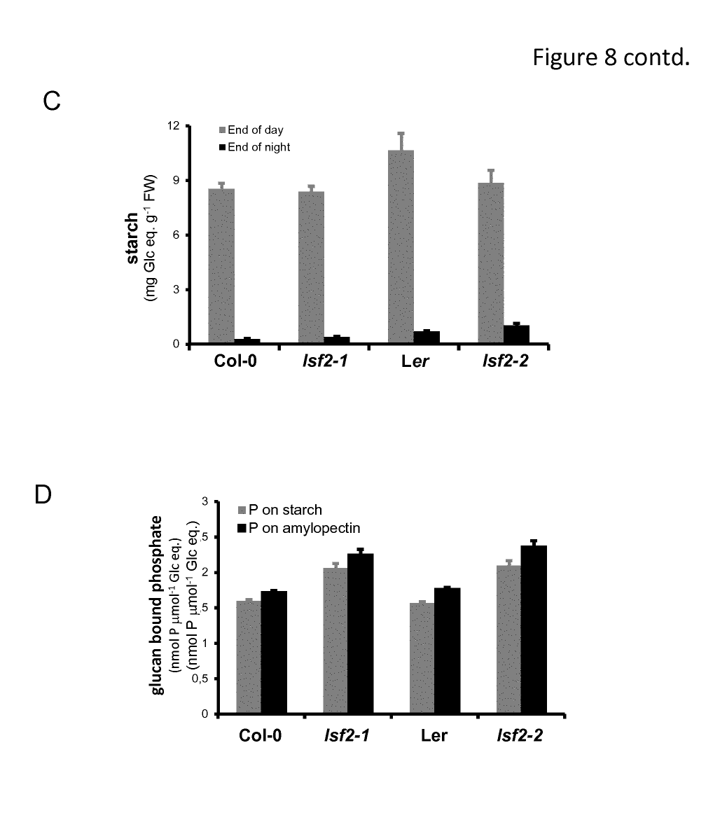

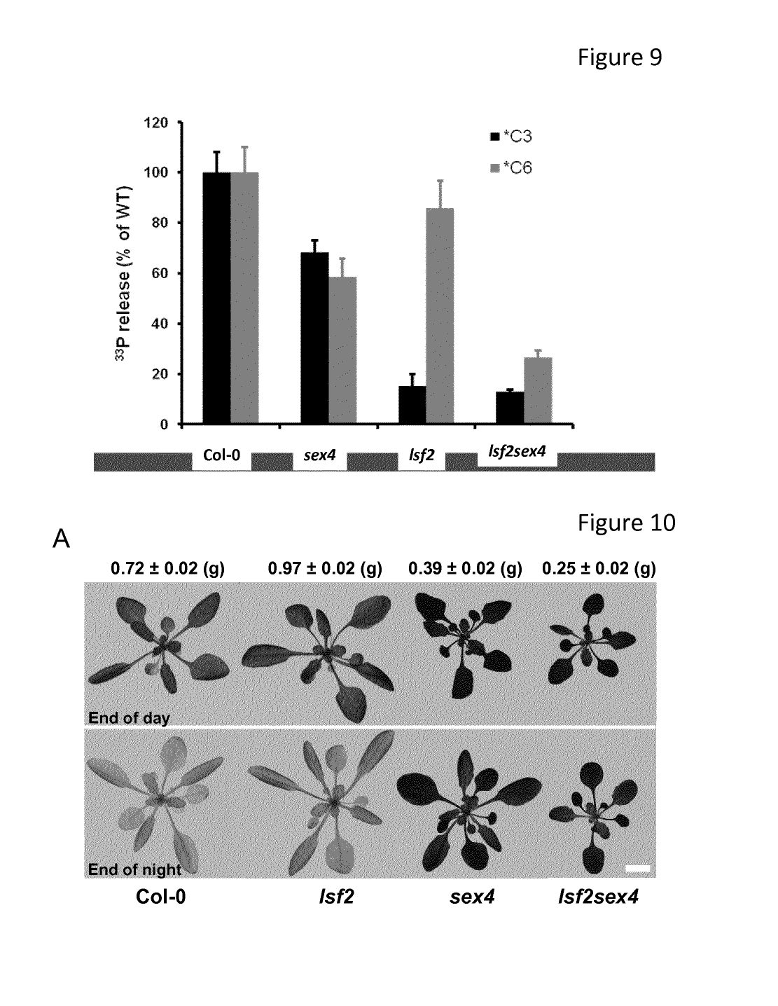

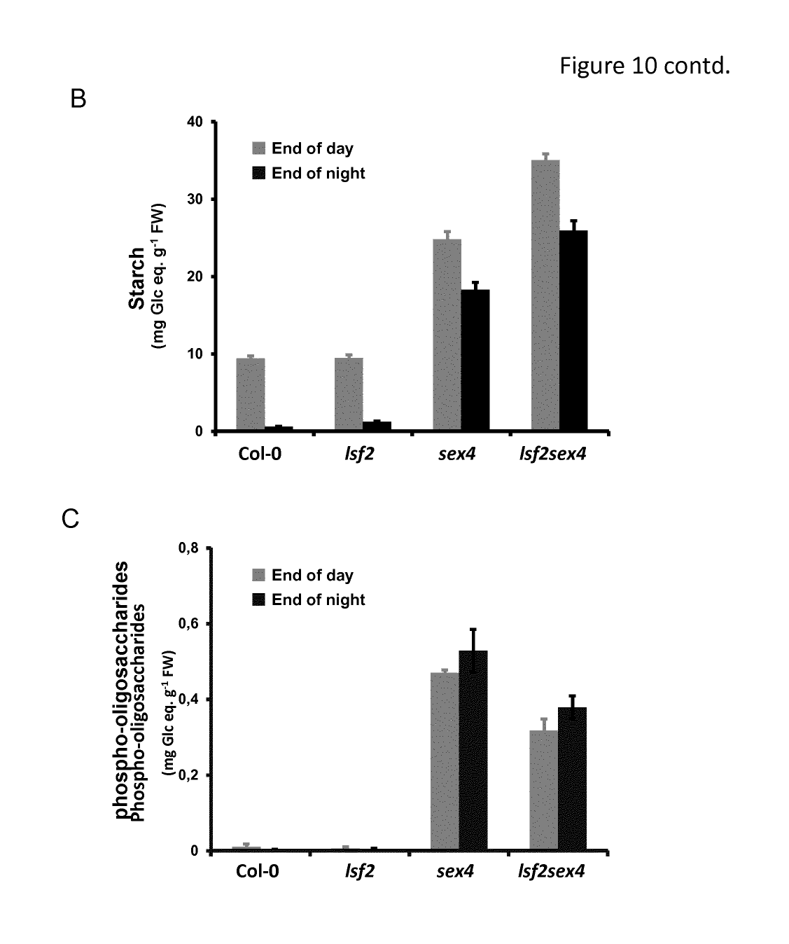

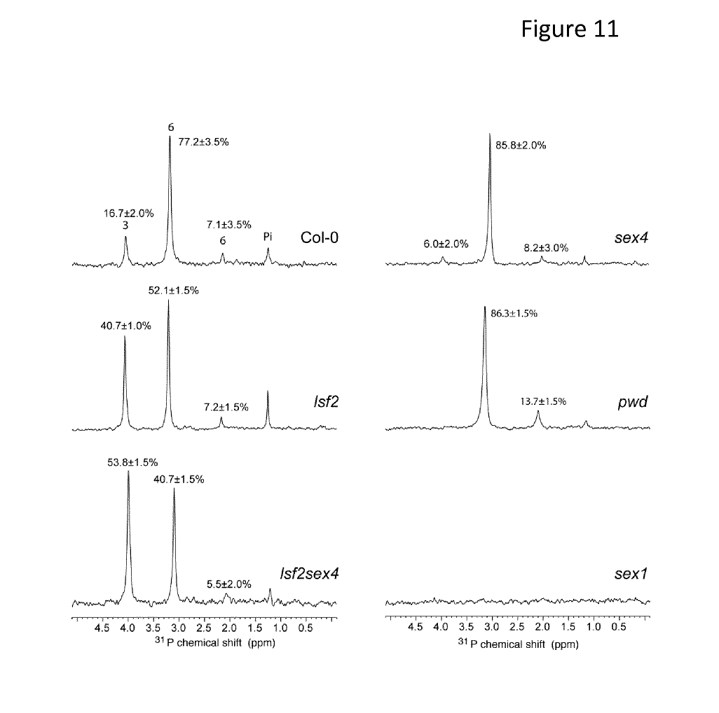

BLAST searches (http://blast.ncbi.nlm.nih.gov) of the Arabidopsis genome revealed two loci encoding proteins with high sequence similarity to SEX4-LSF1 and LSF2 (FIG. 1A, FIGS. 2 A and B; (Comparot-Moss et al., 2010, Plant Physiol. 152, 685-697). LSF2 encodes a 282-amino acid protein with a predicted molecular weight (Mw) of 32.1 kDa (http://www.isb-sib.ch/). The LSF2 protein contains a predicted 61-amino acid chloroplast transit peptide (cTP; Chlor P and TargetP prediction, Emanuelsson et al., 1999, 2000), a Dual Specificity Phosphatase (DSP) domain (residues 85-247) and a C-terminal domain (CT, residues 248-282). The CT extension is not observed in other members of the DSP family but is conserved in LSF1 and LSF2 orthologs from other plant species.

The DSP of LSF2 possesses the canonical DSP active site signature residues HCxxGxxRA/T (FIG. 2A; Yuvaniyama et al., 1996, Science 272, 1328-1331). Interestingly, LSF2 does not possess the carbohydrate binding module (CBM; FIG. 1A) located between the DSP and CT domains in both SEX4 and LSF1, nor does it possess the PDZ-like putative protein-protein interaction domain identified in LSF1 (FIG. 1A; Fordham-Skelton et al., 2002, Plant J. 29, 705-715).

The recently-determined structure of SEX4 provides a molecular basis for understanding its glucan phosphatase function (Vander Kooi et al., 2010, Proc. Natl. Acad. Sci. USA 107, 15379-15384). The DSP domain and CBM interact to form an integral structural unit. The CT domain contacts both the DSP and the CBM domain, and is essential for the folding and solubility of recombinant SEX4 (Vander Kooi et al., 2010). The overall similarity between LSF2 and SEX4 sequences allowed us to model the structure of LSF2 (FIGS. 1B and C, FIG. 2A). Due to the absence of the CBM, the predicted LSF2 structure is more compact than SEX4. Like SEX4, the two α-helices in the CT domain are predicted to wrap around the DSP and cradle the final helix (α8) while making contact with multiple helices (α5, α6, α7; FIG. 1C). To determine whether the CT domain of LSF2 is important for its function, we generated a set of constructs to express LSF2 in Escherichia coli. We previously demonstrated that we could purify full-length SEX4, but it was far more soluble when we removed the cTP (Vander Kooi et al., 2010). Similarly, we found that we could purify full-length LSF2, but we obtained significantly more protein if we removed the cTP at residue Lys65 (Δ65LSF2) (FIG. 1D, FIG. 2C). However, when we deleted the CT domain from Δ65LSF2, the protein was entirely insoluble (FIG. 1D, Δ65LSF2 ΔCT). Similar results were obtained with LSF2ΔCT (FIG. 2C). Thus, the C-terminal domain is necessary for soluble expression of LSF2, as for SEX4.

To discover whether LSF2 is chloroplastic, we examined the subcellular localization of the protein by transiently expressing an LSF2-GFP fusion protein in Arabidopsis protoplasts (FIG. 1E). Free GFP was used as a control and was found in the cytoplasm, whereas the LSF2-GFP fusion protein was localized in the chloroplast. This is consistent with the prediction of a cTP in the LSF2 protein sequence.

Next, we investigated LSF2 expression patterns using a 6-glucuronidase (GUS) transcriptional reporter construct in which 1.5 kb of the LSF2-promoter region upstream of LSF2 translation start site was fused to the GUS gene, resulting in the LSF2pro::GUS fusion. The fusion construct was transformed into wild-type plants and the GUS activity was analyzed in three independent transgenic T2 lines. GUS activity was found in all organs, especially in green tissues, which represent sites of starch storage, and in the vasculature (FIG. 3 A-G). Analysis of publicly accessible transcriptome data was broadly consistent with the GUS activity results (FIG. 3H). Incubation of seedlings containing the LSF2pro::GUS construct in the dark for 72 h revealed decreased LSF2 expression (FIG. 3G). Again, this result was consistent with publicly-accessible transcriptome data showing that LSF2 expression fluctuates diurnally, with transcript abundance declining gradually during the night to a low level, followed by a rapid increase during the first hours of the day (FIG. 3I). This expression pattern is similar to SEX4 (FIG. 3I) and other genes encoding enzymes of starch metabolism, suggesting they are coordinately regulated (Smith et al., 2004, Plant Physiol. 136, 2687-2699). Searching the AtProteome database (Baerenfaller et al., 2008, Science 320, 938-941; http://fgcz-atproteome.unizh.ch/) also provided evidence for the presence of LSF2 predominantly in green tissues (FIG. 3J).

Homologs of LSF2 are found in vascular plants, mosses and in green algae. Maximum likelihood (ML) and Bayesian analyses of 150 unambiguously aligned characters of the DSP domain support the relationship of SEX4, LSF1 and LSF2 (100% ML bootstrap and a posterior probability of 1.0; FIG. 2). The LSF1 proteins, which are absent from green algae, cluster at the base of the SEX4 and LSF2 sister clades (100% ML bootstrap and posterior probability of 1.0). No phosphatase activity has so far been attributed to LSF1. The divergence of the DSP from SEX4 and LSF2 may suggest that it has acquired a new function (Comparot-Moss et al., 2010, Plant Physiol. 152, 685-697; Umhang, submitted). Analysis of the Arabidopsis genes reveals distinct exon-intron structures (FIG. 4).

However, two exon-intron boundaries are conserved between members of the gene family—one within the DSP of all three genes and the other in the CBM domains of SEX4 and LSF1. Along with the similar domain organization in SEX4 and LSF1, this supports a common origin of the CBM in SEX4 and LSF1, indicating its presence in their common ancestor.

### Example 2

### LSF2 Dephosphorylates Amylopectin and Soluble Phosphorylated Glucans

The expression of LSF2 in green tissues, its localization in the chloroplast, its similarity to SEX4 and its co-ordinated expression with other starch metabolizing enzymes all suggest that it may be a glucan phosphatase involved in transitory starch metabolism. To determine if LSF2 is a phosphatase, we tested whether the purified recombinant protein could dephosphorylate the artificial substrate para-nitrophenyl phosphate (p-NPP—a universal chromogenic substrate for acid and alkaline phosphatases). LSF2 was active against p-NPP (FIG. 5A) and had a similar specific activity and kinetic properties to SEX4 (Gentry et al., 2007, J. Cell Biol. 178, 477-488). Within the DSP active site motif HCxxGxxRA/T (Yuvaniyama et al., 1996, Science 272, 1328-1331) the conserved cysteine is essential for activity. Mutation of the corresponding cysteine in LSF2 (C193) to serine abolished its activity against p-NPP (FIG. 5A, LSF2 C/S), analogous to the C198S mutation in SEX4 (Gentry et al., 2007, J. Cell Biol. 178, 477-488). Next, we tested whether LSF2 was able to use phosphorylated glucans as substrates. Potato amylopectin is phosphorylated on approximately 1 in every 300 glucose residues (Blennow et al., 2002), while the soluble phospho-oligosaccharides that accumulate in sex4 (which have a degree of polymerization between 4 and 20) are singly or doubly phosphorylated (Kotting et al., 2009, Plant Cell 21, 334-346). We incubated recombinant LSF2 either with solubilized amylopectin or with purified phosphooligosaccharides and quantified the amount of phosphate released using the malachite green assay (Werner et al., 2005, Arch. Microbiol. 184, 129-136). In each case, recombinant SEX4 was utilized as a positive control. As reported previously, SEX4 displayed robust phosphatase activity towards both substrates (FIG. 3B; Gentry et al., 2007; Kotting et al., 2009, Plant Cell 21, 334-346). LSF2 could also liberate phosphate from both glucan substrates, although to a lesser extent than SEX4 (FIG. 5B). As predicted, mutation of the active site cysteine to serine abolished LSF2 activity. Collectively, these data show that LSF2 possesses a functional DSP domain and is capable of dephosphorylating glucan substrates even though it lacks a CBM.

### Example 3

### LSF2 Specifically Dephosphorylates C3-Glucosyl Residues of Starch In Vitro

The two dikinases GWD and PWD phosphorylate the C6- or the C3-positions of glucosyl units in amylopectin respectively (Ritte et al., 2006, FEBS Lett. 580, 4872-4876). While SEX4 is able to hydrolyze both C6- and C3-bound phosphate, we considered the possibility that LSF2 might be specific for one or the other position. To test this, we phosphorylated purified sex1 starch granules (which are phosphate free; Yu et al., 2001, Plant Cell 13, 1907-1918) in vitro using recombinant potato GWD and recombinant Arabidopsis PWD sequentially. By using [β-³³P]-ATP as a substrate in one dikinase reaction and unlabeled ATP in the other, ³³P-labelled phosphate was introduced at either the C6- or the C3-position. In all cases, the starch granules were phosphorylated at both locations (see Material and Methods for details). After incubation of the recombinant proteins with the ³³P-labelled starches, the released ³³P was determined. The incubation times were short (i.e. 5 min), such that the rates of hydrolysis from both C6- and C3-positions were linear. The amounts of the two phosphatases were adjusted to equal hydrolytic activity on p-NPP (SEX4 0.05 μg; LSF2 0.2 μg). Recombinant SEX4 efficiently released phosphate from both positions, but twice as much from the C6- as from the C3-position (FIG. 5C; Hejazi et al., 2010, Plant Physiol. 152, 711-722). This may reflect a preference for the 06-phosphate esters. However, as less ³³P was incorporated into the C3-positions as compared to the C6-positions (20.41 versus 47.72 pmol P mg⁻¹ starch), it may also reflect a lower frequency of C3-bound phosphate groups on the granule surface. When LSF2 was incubated with starch granules labeled with ³³P at the C6-position, no radioactivity was released. Conversely, LSF2 efficiently hydrolyzed the phosphate esters at the C3-position. Again, mutation of the conserved cysteine residue abolished LSF2 activity (FIG. 5C).

Thus, LSF2 is unique as it is highly specific for the C3-position of glucosyl residues of starch even if, under saturating conditions, it has a low capacity to dephosphorylate some C6-esters.

### Example 4

### LSF2 Binds Starch Despite Lacking a CBM and is Present Inside Starch Granules

To test the starch-binding capacity of LSF2, we performed a starch binding assay with purified recombinant LSF2 and SEX4, and a commercially available alkaline phosphatase (AP) as a nonbinding control (FIG. 5D). The recombinant proteins were incubated with amylose-free potato starch and the starch was washed with assay medium to remove all the unbound proteins (FIG. 5D, lane W). All the SEX4 protein bound to starch, as demonstrated by the absence of the protein in the soluble fraction after starch pull-down (FIG. 5D). This result is consistent with previous reports that CBM48s confer a high affinity towards starch (Niittyla et al., 2006, J. Biol. Chem. 281, 11815-11818; Gentry et al., 2007, J. Cell Biol. 178, 477-488). The LSF2 protein was also able to bind to starch, but the affinity may be lower than that of SEX4, as demonstrated by the fact that some soluble LSF2 was still visible on silver-stained SDS-PAGE gels (FIG. 5D). As expected, alkaline phosphatase did not bind to starch. These data show that despite lacking a CBM, recombinant LSF2 can still bind to starch, perhaps through secondary binding sites within or adjacent to the catalytic domain.

To confirm that endogenous LSF2 binds starch, we incubated potato starch with protein extracts from Arabidopsis leaves and analyzed the bound proteins by SDS-PAGE (FIG. 7, “Binding”) and LC-MS/MS. Amongst the identified peptides derived from the bound proteins, one was unambiguously assigned to LSF2 in gel slices corresponding to the predicted 32 kDa Mw of LSF2 (SEQ ID NO: 30). Next, we isolated starch from Arabidopsis leaves harvested at the end of the light period and analyzed proteins bound to the starch granule surface (External) and proteins encapsulated within the starch granule (FIG. 7, “Internal”). One LSF2 peptide was identified in the external fraction (SEQ ID NO: 31) and three in the internal fraction (SEQ ID NOs: 32 to 34). These proteomics data support our in vitro binding experiment with the recombinant LSF2 protein.

### Example 5

### Loss of LSF2 Results in Elevated Glucan-Bound Phosphate Levels

To study the function of LSF2 in vivo, we identified two independent Arabidopsis insertion mutants at the LSF2 locus (FIG. 4) and designated them Isf2-1 (Sail_{—}595F04) and Isf2-2 (GT10871). We obtained homozygous lines and confirmed the positions of the insertions by PCR and DNA sequencing (in exon 2 at bp+510 from the start codon and exon 4 at bp+1016 for Isf2-2 and Isf2-1, respectively). Quantitative RT-PCR revealed that both insertions prevented normal LSF2 gene expression relative to the corresponding wild types (FIG. 8A).

To determine the contribution of LSF2 to total glucan phosphatase activity in vivo, we incubated starch granules labeled with ³³P at either the C6- or the C3-position with crude extracts from leaves of the Isf2 mutants or their respective wild types (FIG. 9 and FIG. 8B).

Consistent with our in vitro assay, Isf2 extracts released 80% less phosphate from the C3-position than extracts of wild-type leaves, whereas phosphate release from the C6-position was unaltered. The residual C3-phosphatase activity of Isf2 extracts can be attributed to the activity of SEX4 or other phosphatases in Isf2 extracts.

Leaves of Isf2-1 and Isf2-2 and their respective wild types were harvested at the end of the day and the end of the night. No differences in leaf starch content were revealed in either mutant compared with their wild types by qualitative iodine staining (FIG. 10A) or by quantitative measurements after digestion of starch to glucose (FIG. 10B and FIG. 8C). Thus, the loss of LSF2 does not prevent a normal rate of transitory starch degradation, at least under our growth conditions. We determined whether Isf2 mutants had altered glucan-bound phosphate by measuring total phosphate levels of leaf starch extracted at the end of the day, and by measuring whether Isf2 plants contained soluble phospho-oligosaccharides. Total starch-bound phosphate was significantly elevated in both Isf2 alleles compared to their wild types (see Table 1 and FIG. 8D), supporting the idea that LSF2 dephosphorylates starch in vivo. However, no soluble phospho-oligosaccharides were detected in extracts of Isf2 leaves, whereas they were present in high amounts in sex4 extracts (FIG. 10C; Kötting et al., 2009, Plant Cell 21, 334-346).

TABLE 1
Leaf starch was purified from pools of hundreds of 4-week-old
plants harvested at the end of the light period. The amylopectin
content was determined to be 92.6% ± 0.2% for the wild type,
91.4% ± 0.1% for lsf2, 79.1% ± 0.4% for sex4. Starch-
bound phosphate from the same preparation was determined using
the malachite green assay (see Material and Methods for details).
The values show the results of one representative experiment with
the SE of three technical replicates (p value < 0.05). Similar
results were obtained in a second independent experiment.

(nmol phosphate μmol⁻¹ Glc eq.)

| | Starch | Amylopectin |
|---|---|---|
| Col-0 | 1.35 ± 0.037 | 1.47 ± 0.037 |
| lsf2 | 1.8 ± 0.04 | 1.97 ± 0.04 |
| sex4 | 1.09 ± 0.036 | 1.33 ± 0.036 |

### Example 6

### Isf2 Starch Contains High Levels of C3-Bound Phosphate

We reasoned that the elevated glucan-bound phosphate of the Isf2 starch could represent phosphate bound specifically to the C3-position. Therefore, we determined the chemical nature of the phosphate in starch isolated from leaves of wild-type and Isf2 plants by ³¹P Nuclear Magnetic Resonance (NMR) analysis (FIG. 11, Table 2).

TABLE 2
Acquisition parameters for 2D NMR spectroscopy.

| experiment | sample | sw | sw1 | ns | ni | at | mix | np | D1 |
|---|---|---|---|---|---|---|---|---|---|
| 1D ³¹P | Glc-3P and Glc-6P | 50.7 | | 128 | | 1328 | | 32768 | 3 |
| 1D ³¹P | hydrolized potato starch | 50.7 | | 14336 | | 1328 | | 32768 | 3.5 |
| 2D ³¹P-¹HHSQC | Glc-3P and Glc-6P | 13.9 | 8.0 | 2 | 256 | 123 | 40 | 2048 | 1.5 |
| 2D ³¹P-¹HHSQC | hydrolized potato starch | 13.9 | 20.0 | 128 | 128 | 123 | 40 | 2048 | 1.5 |
| 2D TOCSY | Glc-3P and Glc-6P | 10.0 | 10.0 | 32 | 512 | 170 | 120 | 2048 | 1.0 |
| 2D TOCSY | hydrolized potato starch | 10.0 | 10.0 | 32 | 512 | 170 | 120 | 2048 | 1.3 |
| 2D DQF-COSY | potato starch hydrolysate | 10.0 | 10.0 | 56 | 670 | 340 | | 4096 | 1.0 |

Abbreviations used: sw: spectral width in the directly detected dimension in ppm; sw1: spectral width in the indirectly detected dimension in ppm; ns: number of scans; ni: number of increments; at: acquisition time in ms; mix: mixing time for TOCSY or length of INEPT block in ms; np: number of points in the directly detected dimension; d1: interscan delay in s.

Starch samples were digested with α-amylase and amyloglucosidase and the products of digestion were subjected to MALDI/MS/MS analysis prior to NMR analysis (see Material and Methods). MALDI TOF mass spectra revealed the presence of signals consistent with phosphooligosaccharides, varying from three to 16 hexoses plus one or two phosphates. Although the phospho-oligosaccharide mixture is heterogeneous in terms of polymerization state, the ³¹P chemical shifts are mainly influenced by the local environment (e.g. formed by three consecutive glucoses), and are similar in phospho-oligosaccharides of different lengths.

A 1-D ³¹P spectrum of wild-type samples revealed four signals corresponding to four phosphate species. The type of linkage to glucose can be determined by analyzing through-bond long-range coupling constants (³J_{HP}) between ¹H and ³¹P with a ³¹P-¹H HSQC (Heteronuclear Single-Quantum Correlation) spectrum (Table 2). In the case of O3-attachment, one signal correlating H3 and P as in Glc-3P is expected, whereas phosphate at O6 leads to two signals correlating H6 and H6′ with P. In the starch spectrum, signal 1 on the left shows one ¹H-³¹P correlation (Table 2) and can thus be assigned as O3 attachment, signals 2 and 3 show correlations to two protons and can be assigned as O6 attachment. Signal 4 does not show any ¹H-³¹P correlation and likely originates from inorganic orthophosphate (Table 2). Our results build on previous NMR analyses (Ritte et al., 2006, FEBS Lett. 580, 4872-4876), but allow better separation of the signals at pH 6.0 enabling us to assign the previously unassigned signal 2 to a second C-6 phosphate species.

The ratio of C3- to C6-bound phosphate in wild-type starch was approximately 1:5 (FIG. 11, Table 3), consistent with previous reports (Ritte et al., 2006, FEBS Lett. 580, 4872-4876; Haebel et al., 2008, Anal. Biochem. 379, 73-79). We analyzed pwd, sex1 and sex4 starches as controls. As expected, C3-phosphate was reduced to the limits of detection in pwd starch, while sex1 starch was phosphate free (FIG. 11; Ritte et al., 2006, FEBS Lett. 580, 4872-4876). In sex4 starch, the ratio of C3- to C6-bound phosphate was appreciably lower than the wild type (0.06 versus 0.20; FIG. 11, Table 3; Comparot-Moss et al., 2010, Plant Physiol. 152, 685-697). Remarkably, in starch from both Isf2 mutant alleles the proportion of C3-bound phosphate was increased to levels not previously reported (40.7% and 37.9%, for Isf2-1 and Isf2-2, respectively; FIG. 11, Table 3). These experiments suggest that the elevated levels of total glucan-bound phosphate in the Isf2 starch results from an accumulation of C3-bound phosphate. Thus, LSF2 makes a major contribution to dephosphorylation at the C3-position in vivo and appears to be only partially redundant with SEX4.

TABLE 3
C3- and C6-bound phosphate contents of leaf starch in wild-type
and mutant plants. Leaf starch was purified from plants harvested
at the end of the light period and total starch-bound phosphate
was determined by the malachite green assay. The relative amounts
of C3- and C6-bound phosphate were determined based on the peak
areas of the corresponding 31P NMR spectra (see FIG. 8).

| | C6-bound | C3-bound | |
|---|---|---|---|
| | nmol μmol⁻¹ Glc eq. | nmol μmol⁻¹ Glc eq. | Ratio C3/C6-bound |
| Col-0 | 1.32 ± 0.05 | 0.27 ± 0.03 | 0.20 |
| sex4 | 1.24 ± 0.03 | 0.08 ± 0.03 | 0.06 |
| lsf2-1 | 1.25 ± 0.03 | 0.86 ± 0.02 | 0.69 |
| Ler-0 | 1.47 ± 0.03 | 0.3 ± 0.04 | 0.2 |
| lsf2-2 | 1.47 ± 0.03 | 0.9 ± 0.04 | 0.61 |

### Example 7

### Plant Transformation Vector to Reduce Expression of LSF-2 in Wheat

Unless stated otherwise in the Examples, all recombinant DNA techniques in this and the following example are carried out according to standard protocols as described in Sambrook et al. (1989) Molecular Cloning: A Laboratory Manual, Second Edition, Cold Spring Harbor Laboratory Press, NY and in Volumes 1 and 2 of Ausubel et al. (1994) Current Protocols in Molecular Biology, Current Protocols, USA. Standard materials and methods for plant molecular work are described in Plant Molecular Biology Labfax (1993) by R. D. D. Croy, jointly published by BIOS Scientific Publications Ltd (UK) and Blackwell Scientific Publications, UK. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany. Using standard recombinant DNA techniques the plasmid pTMV398 was constructed and used to transform heat shock competent Agrobacterium tumefaciens strain AG L1.

The vector pTMV398 is derived from pGSC1700 (Cornelissen and Vandewiele, 1989, Nucleic Acids Research, 17, 19-25).

The genetic elements are described in Table 4 below.

TABLE 4
Description of the genetic elements of pTMV398.

| Nt Positions | Orientation | Origin |
|---|---|---|
| 1-25 | | RB: right border repeat from the T-DNA of Agrobacterium |
| | | tumefaciens (Zambryski, 1988, Annual Review of Genetics, 22, 1-30 |
| 26-88 | | Synthetic polylinker derived sequences |
| 89-506 | Clockwise | P35S2: sequence including the promoter region of the Cauliflower |
| | | Mosaic Virus 35S transcript (Odell et al., 1985, Nature, 313, 810-812) |
| 507-580 | Clockwise | 5′cab22L: sequence including the leader sequence of the |
| | | chlorophyl a/b binding protein gene of Petunia hybrida (Harpster et |
| | | al., 1988, Molecular and General Genetics, 212) |
| 581-830 | Clockwise | lsf2Ta-hpf1: part of the coding sequence of the phosphoglucan |
| | | phosphatase LSF2 gene of Triticum aestivum (wheat) (Santelia et |
| | | al., 2011, The Plant Cell, 23, 4096-4111) |
| 831-1638 | Clockwise | intron2 pdkFt: second intron of the pyruvate orthophosphate |
| | | dikinase gene of Flaveria trinervia (Clustered Yellowtops) (Rosche |
| | | and Westhoff, 1995, Plant Molecular Biology, 29, 663-678) |
| 1639-1888 | Counter | lsf2Ta-hpf1: part of the coding sequence of the phosphoglucan |
| | clockwise | phosphatase LSF2 gene of Triticum aestivum (wheat) (Santelia et |
| | | al., 2011) |
| 1889-2174 | Clockwise | 3′nos: sequence including the 3′ untranslated region of the |
| | | nopaline synthase gene from the T-DNA of pTiT37 (Depicker et |
| | | al., 1982, Journal of Molecular and Applied Genetics, 1, 561-573) |
| 2175-2216 | | Synthetic polylinker derived sequences |
| 2217-2250 | Clockwise | lox: sequence including the 34 bp recognition sequence for the |
| | | Cre recombinase of bacteriophage P1 (Hoess and Abremski, |
| | | 1985, Journal of Molecular Biology, 181, 351-362) |
| 2251-3087 | Clockwise | P35S3: sequence including the promoter region of the Cauliflower |
| | | Mosaic Virus 35S transcript (Odell et al., 1985) |
| 3088-3639 | Clockwise | bar: coding sequence of the phosphinothricin acetyltransferase |
| | | gene of Streptomyces hygroscopicus (Thompson et al., 1987, EMBO |
| | | Journal, 6, 2519-2523). |
| 3640-3923 | Clockwise | 3′nos: sequence including the 3′ untranslated region of the |
| | | nopaline synthase gene from the T-DNA of pTiT37 (Depicker et |
| | | al., 1982) |
| i | Clockwise | lox: sequence including the 34 bp recognition sequence for the |
| 3924-3957 | | Cre recombinase of bacteriophage P1 (Hoess and Abremski, |
| | | 1985) |
| 3958-4019 | | Synthetic polylinker derived sequences |
| 4020-4044 | | LB: left border repeat from the T-DNA of Agrobacterium |
| | | tumefaciens (Zambryski, 1988) |
| 4045-4349 | | Ti-plasmid sequences of pTiAch5 flanking the left border repeat |
| | | (Zhu et al., 2000, Journal of Bacteriology, 182 (14), 3885-3895). |
| 4350-6134 | Counter | aadA: fragment including the aminoglycoside adenyltransferase |
| | clockwise | gene of Escherichia coli (Fling et al., 1985, Nucleic Acids Research, |
| | | 13, 7095-7106). |
| 6135-8924 | | ORI pVS1: fragment including the origin of replication from the |
| | | Pseudomonas plasmid pVS1 for replication in Agrobacterium |
| | | tumefaciens (Hajdukiewicz et al., 1994, PI Mol Biology, 25, 989-994). |
| 8925-10081 | | ORI ColE1: fragment including the origin of replication from the |
| | | plasmid pBR322 for replication in Escherichia coli (Bolivar et al., |
| | | 1977, Gene, 2, 95-113). |
| 10082-10287 | | Ti- plasmid sequences of pTiAch5 flanking the right border repeat |
| | | (Zhu et al., 2000) |

### Example 8

### Agrobacterium-Mediated Transformation of Wheat

### Donor Plant Growth

Spring wheat donor plants were grown in 17 cm pots under controlled-environment conditions (25° C. day/20° C. night, 16 h day, 250 μmole/m²/sec at pot level). Plants were given an N—P-K 11:11:11 fertilizer supplement at a rate of 3 g/pot. Where possible donor plants were grown without the application of pesticides or fungicides.

### Isolation and Sterilization of Immature Embryos

Immature seeds (containing embryos of 2-3 mm in size) were harvested 10-12 weeks after sowing. After peeling of the outer husk with fine forceps the immature seeds were sterilized by incubating for 1 min in 70% v/v ethanol, followed by 15 min agitation in bleach solution (1.3% active chlorine) and finally washed 3× with sterile water.

Infection with Agrobacterium

Transformation was performed essentially as described by Wu et al. (Plant Cell Rep 21 (2003): 659-668). Agrobacterium strain AGL1 was grown as a 20 ml preculture in MGL medium (Tingay et al., Plant J 11 (1997): 1369-1376) without selection (overnight, 150 rpm, 28°). Shortly before use the OD₆₀₀ was measured and the final density of bacteria adjusted to OD₆₀₀=0.5-1.0 in inoculation buffer (supplemented with 200 μM acetosyringone). Immature embryos were carefully excised (+/−embryo axis) under a stereo-microscope and transferred (scutellum-side up) to 5.5 cm plates containing co-cultivation medium (25-50 embryos/plate). 1-2 ml of the Agrobacterium suspension was then added slowly to each plate to cover the embryos. After 15-30 min incubation at room temperature the embryos were removed (blotted dry to remove excess liquid) and transferred (same orientation) to fresh co-cultivation medium. Embryos were co-cultivated with Agrobacterium for 2-3 days in the dark.

### Selection and Regeneration

Following co-cultivation the immature embryos were transferred to 9 cm dishes containing callus induction medium. All media subsequently used in the procedure contain 160 mg/l of the antibiotic Timentin to control Agrobacterium growth. After 2 weeks of culture in the dark calli were divided and transferred to fresh callus induction medium. After a further 2 weeks of culture the embryogenic calli were transferred to plates containing regeneration medium and transferred to the light (16 h day/night). Regenerating calli were picked and transferred after 2-3 weeks to regeneration medium containing PPT selection (2.5-5 mg/l). Shoots showing persistent growth on PPT (with repeated subculture where necessary) were transferred to magenta boxes for rooting. AgraStrip® LL Strips (Romer Labs®, Inc) were used to confirm bar gene expression (detection of PAT protein in leaf tissue) in transformants prior to transfer to the greenhouse.

1. Genetically modified plant cell, comprising a reduced activity of at least one LSF-2 protein in comparison with corresponding wild type plant cells that have not been genetically modified.

2. Genetically modified plant cell according to claim 1, wherein the genetic modification comprises an introduction of at least one foreign nucleic acid molecule into the genome of the plant cell.

3. Genetically modified plant cell according to claim 1, wherein the said foreign nucleic acid molecule is selected from the group consisting of
a) DNA molecules, which code at least one antisense RNA, which effects a reduction in the expression of at least one endogenous gene, which encodes an LSF-2 protein;
b) DNA molecules, which by means of a co-suppression effect lead to the reduction in the expression of at least one endogenous gene, which encodes an LSF-2 protein;
c) DNA molecules, which code at least one ribozyme, which splits specific transcripts of at least one endogenous gene, which encodes an LSF-2 protein;
d) DNA molecules, which simultaneously encode at least one antisense RNA and at least one sense RNA, wherein the said antisense RNA and the said sense RNA form a double-stranded RNA molecule, which effects a reduction in expression of at least one endogenous gene, which encodes an LSF-2 protein (RNAi technology);
e) Nucleic acid molecules introduced by in vivo mutagenesis, which lead to a mutation or an insertion of a heterologous sequence in at least one endogenous gene encoding an LSF-2 protein, wherein the mutation or insertion effects a reduction in expression of a gene encoding an LSF-2 protein or results in synthesis of inactive LSF-2 protein;
f) Nucleic acid molecules, which encode an antibody, wherein the antibody results in a reduction in activity of an LSF-2 protein due to the bonding to an LSF-2 protein,
g) DNA molecules, which comprise transposons, wherein integration of said transposons leads to a mutation or an insertion in at least one endogenous gene encoding an LSF-2 protein, which effects a reduction in expression of at least one gene encoding an LSF-2 protein, or results in synthesis of inactive LSF-2 proteins; or
h) T-DNA molecules, which, due to insertion in at least one endogenous gene encoding an LSF-2 protein, effect a reduction in expression of at least one gene encoding an LSF-2 protein, or result in synthesis of inactive LSF-2 protein.

4. Plant cell according to claim 1, which synthesises a modified starch in comparison with corresponding wild type plant cells that have not been genetically modified.

5. Plant cell according to claim 4 wherein the starch is comprises an increased amount of starch phosphate bound in the C-3 position of the glucose molecules in comparison to starch isolated from corresponding non-genetically wildtype plant cells.

6. Plant cell according to claim 4, wherein the modified starch wherein the ratio of starch phosphate bound in the C-3 position to C-6 position of the glucose molecules is increased in comparison to the ratio of phosphate bound in the C-3 position to C-6 position of the glucose molecules in starch isolated from corresponding non-genetically wildtype plant cells.

7. Plant comprising plant cells according to claim 1.

8. Propagation material of a plant according to claim 7.

9. Harvestable plant parts of a plant according to claim 7.

10. A method for producing a genetically modified plant wherein
a) a plant cell is genetically modified, whereby the genetic modification leads to reduction of activity of an LSF-2 protein in comparison with corresponding wild type plant cells that have not been genetically modified;
b) a plant is regenerated from plant cells from a); and
c) optionally, further plants are produced with the help of the plants according to b).

11. Modified starch obtainable from a genetically modified plant cell according to claim 1.

12. Modified starch according to claim 11 wherein the starch comprises an increased amount of starch phosphate bound in the C-3 position of the glucose molecules in comparison to starch isolated from corresponding non-genetically wildtype plant cells.

13. A method for manufacturing a modified starch comprising extracting the starch from a plant cell according to claim 1.

14. A method for manufacturing derived starch, comprising deriving a modified starch according to claim 11.

15. Derived starch obtainable by means of a method according to claim 14.

16. A recombinant nucleic acid molecule comprising a heterologous promoter, which initiates transcription in a plant cell, and an nucleic acid sequence or a complementary thereof encoding an LSF-2 protein.

17. Composition comprising a recombinant nucleic acid molecule as defined in claim 16.

18. A protein comprising activity of an LSF-2 protein, wherein the LSF-2 protein de-phosphorylates phosphate residues bound in the C-3 position of the glucose molecules in the starch but does not de-phosphorylate phosphate residues bound in the C-6 position of the glucose molecules in the starch.
